(19) United States
(12) Patent Application Publication
(10) Pub. No.: US 2011/0240034 A1
(43) Pub. Date: Oct. 6, 2011

(54) SECUREMENT SYSTEM FOR AN ENDOCTRACHEAL TUBE

(75) Inventor: Paul Ciccone, Social Circle, GA (US)

(73) Assignee: Venetec International ,Inc., Covington, GA (US)

(21) Appl. No.: 12/596,834

(22) PCT Filed: Sep. 16, 2008
(86) PCT No.: PCT/US2008/076558

§ 371 Date: Oct. 20, 2009

(51) Int. Cl. A61M 16/04

(52) U.S. Cl. 128/207.17

(57) ABSTRACT

A securement system secures an endotracheal tube or other medical article in position upon a patient and arrests movement of the endotracheal tube. The securement system includes a support member and a retainer. The retainer defines a channel configured to receive the endotracheal tube. A head securement member attaches the support member to the head of the patient. The securement system can include a soft gel for placement against the patient's skin and well as a track for securing the endotracheal tube at a plurality of locations relative to the support member. Advantageously, the securement system can maintain an endotracheal tube in position upon a patient and allow access to the patient's oral cavity.

### BACKGROUND

1. Field of the Invention

The present invention relates generally to techniques and systems for securing an endotracheal tube or other medical line to a patient, and to techniques and systems for accessing the oral cavity of the patient when the medical line is secured to the patient.

2. Description of the Related Art

Endotracheal tubes are commonly administered to medical patients, for example to mechanically-ventilated patients. Placement of an endotracheal tube into the throat of a patient is referred to as intubation. Failing to secure the endotracheal tube properly, however, can result in the migration of the tube within the throat of the patient. Such motion is undesirable since outward motion of the tube can result in the tube moving entirely out of the airway of the patient, eliminating its effectiveness and potentially ventilating the patient's gastro-intestinal tract instead of his lungs. Inward motion of the tube is undesirable because it will eventually result in the tube moving down one of the patient's bronchi, preventing air from being ventilated to the other lung. This will quickly lead to the collapse of the unventilated lung. Even slight back and forth motion of the tube within the throat can result in tracheomalacia and ultimately in the weakening or collapse of the trachea, which may require permanent stenting to maintain an open airway. Migration may further result in a form of nosocomial pneumonia.

Nosocomial pneumonia, also referred to as hospital-acquired pneumonia, is a serious complication in mechanically-ventilated patients. Pneumonia is one of the most common nosocomial infections in the United States, and results in substantial morbidity and mortality. The risk for pneumonia is increased by the direct access of bacteria to the lower respiratory tract. This occurs because of leakage around the cuff of an administered endotracheal tube, thereby enabling pooled secretions to enter the trachea. Pneumonia is frequently caused by the aspiration of mouth secretions into the upper airways.

In order to avoid these undesirable consequences, it is common to secure the endotracheal tube in position upon the patient by means of adhesive tape. For instance, a medical practitioner may, after positioning the tube within the throat of the patient, wrap adhesive tape around the tube and tape it to the patient. Various devices are often used as adaptors for endotracheal tubes, and sometimes these devices are taped to the patient instead of directly taping the endotracheal tube to the patient.

Such arrangements present certain problems for a medical practitioner. One difficulty is that once secured, it is generally not convenient to release or adjust the securement of the endotracheal tube. For instance, if an adhesive tape is used to secure the tube to the patient, adjusting or re-securing the tube in such situations requires that the adhesive tape be removed from the patient and the tube, and then fresh adhesive tape be used to properly re-secure the tube upon the patient. Such adhesive tape can be difficult to work with for medical practitioners wearing latex gloves, and contact with the adhesive can introduce tears or microscopic perforations into the gloves, compromising their effectiveness.

Furthermore, the use of adhesive tape may be harmful to the patient. Repeated application and removal of adhesive from the face of a patient can irritate or damage the skin of the patient, which is a particular risk for long-term ventilated patients. In addition, adhesive tape can harbor a significant number of bacteria around a patient's skin. Furthermore, adhesive tape may not effectively prevent repositioning and unplanned extubation of the endotracheal tube. As discussed above, migration of the tube within the throat of the patient is undesirable, and unplanned extubation or repositioning of the tube can cause secretions to leak around an inflation cuff of the tube and thereby leak into a patient's upper airways.

After an endotracheal tube is attached to a patient, access to the patient's mouth may be impaired by the endotracheal tube. However, allowing a medical practitioner access to the patient's mouth, for example to provide oral care, is desirable because ventilated patients are typically unable to care for themselves, and thus are unlikely to exercise proper oral care.

Improper oral care can facilitate the colonization of harmful bacteria in the oral cavity. The mouth is colonized with hundreds of microorganism species. The endotracheal tube provides a surface on which these microorganisms can colonize and establish a biofilm. While concentrations of microorganisms in the oral cavity are generally regulated by competitive inhibition among species, the introduction of a foreign surface such as an endotracheal tube may alter the balance of microorganisms in the oral cavity. Furthermore, the endotracheal tube may provide a pathway for microorganisms to migrate. Even microorganisms that normally colonize in the oral cavity may cause harm to the patient if allowed to migrate, such as to the lower airways.

The problem of bacteria colonization may be exacerbated in an intubated patient because normal immune defense mechanisms are impaired in such patients. For example, saliva production, the ability to swallow, and the cough reflex may all be impaired. Such impairment allows microorganisms increased access to the lower airways.

Medical practitioners, such as nurses or respiratory technicians, are expected to clean a patient's oral cavity at regular intervals, e.g., every four hours. Oral care of an intubated patient, however, is a difficult and time-consuming task for medical practitioners, many of whom are consumed with other high-priority tasks.

Therefore, a need continues to exist for an improved system to secure an endotracheal tube to a medical patient and for a system that allows for improved access to a patient's mouth.

### SUMMARY

One aspect of the present invention thus involves a securement system for an endotracheal tube. The securement system includes a support member having a track and a posterior facing surface configured to inhibit movement of the support member into a person's mouth; and a retainer having a channel configured to receive a portion of the endotracheal tube. The retainer is configured to attach to the track and is movable with respect to the support member in at least one direction.

Another aspect involves a securement system for an endotracheal tube. The securement system includes a track, and a retainer configured to receive a first portion of the endotracheal tube so as to inhibit movement of the endotracheal tube in at least one direction while a second portion of the endotracheal tube is placed in a patient's mouth. The retainer is attached to the track so as to translate along the track without being removed from the track.

Yet another aspect involves a method of securing an endotracheal tube in a first position on a patient so as to facilitate access to the patient's oral cavity. The method includes providing an endotracheal securement device having a support member and a retainer, the retainer defining a channel and being movable with respect to the support member; placing a portion of the endotracheal tube in the channel; and rotating at least a portion of the retainer so as to secure the portion of the endotracheal tube in the channel.

Further aspects, features, and advantages of the present invention will become apparent from the detailed description of certain embodiments that follows.

### BRIEF DESCRIPTION OF THE DRAWINGS

The above mentioned and other features of the invention will now be described with reference to the drawings of several embodiments of the present securement system. The illustrated embodiments of the securement system are intended to illustrate, but not to limit the invention. The drawings contain the following figures:

FIG. 1 is a perspective view of a securement system in accordance with an embodiment of the present invention and shows a support member, a retainer, connection members, and a strap.

FIG. 2 is a perspective view of the support member from FIG. 1.

FIG. 3 is a top view of the support member from FIG. 2.

FIG. 4 is another top view of the support member from FIG. 2 and shows sides of the support member flexed towards each other.

FIG. 5 is a front view of the support member from FIG. 2.

FIG. 6 is a side view of the support member from FIG. 2.

FIG. 7 is a cross-sectional view of the support member from FIG. 2, taken along line 7-7 of FIG. 3.

FIG. 8 is a perspective view of the retainer from FIG. 1.

FIG. 9 is a side view of the retainer from FIG. 8, taken from the side opposite which the retainer is viewed from in FIG. 8.

FIG. 10 is an opposite side view and shows a movable member engaged with an attachment member.

FIG. 11 is a side view of the retainer from FIG. 10 with the movable member disengaged from the attachment member.

FIG. 12 is a top view of the retainer from FIG. 8.

FIG. 13 is a front view of the retainer from FIG. 8.

FIG. 14 is a back view of the retainer from FIG. 8.

FIG. 15A is a cross-sectional view of the retainer taken along the line 15-15 of FIG. 12 and shows the moveable member engaged with the attachment member.

FIG. 15B is a cross-sectional view similar to FIG. 15A except that the moveable member is disengaged from the attachment member.

FIG. 16 is a side view of the retainer from FIG. 8 attached to the support member from FIG. 2.

FIG. 17 is a front view of the retainer from FIG. 8 attached to the support member from FIG. 2 in a first position.

FIG. 18 is a front view of the retainer from FIG. 8 attached to the support member from FIG. 2 in a second position.

FIG. 19 is a top view of the connection member from FIG. 1.

FIG. 20 is a bottom view of the connection member from FIG. 19.

FIG. 21 is a side view of the connection member from FIG. 19.

FIG. 22 is a side view of the connection member from FIG. 19 attached to the support member from FIG. 2.

FIG. 23 is side view of a plurality of connection members attached to the support member from FIG. 2.

FIG. 24 is a top view of the strap from FIG. 1.

FIG. 25 is a bottom view of the strap from FIG. 24.

FIG. 26 is a side view of the strap from FIG. 24.

FIG. 27 is a side view of an intubated patient.

FIG. 28 is a side view of the securement system from FIG. 1 attached to the intubated patient from FIG. 27.

FIG. 29 is a front view of the securement system and intubated patient from FIG. 28 and shows the retainer in a first position.

FIG. 30 is similar to FIG. 29 except that the retainer is in a second position.

FIG. 31 is a perspective view of a securement system in accordance with an embodiment of the present invention and shows a support member, a retainer, connection members, and a harness.

FIG. 32 is a perspective view of the support member from FIG. 31.

FIG. 33 is a front view of the support member from FIG. 32.

FIG. 34 is a side view of the support member from FIG. 32.

FIG. 35 is a perspective view of the retainer from FIG. 31.

FIG. 36 is a side view of the retainer from FIG. 35, taken from the side nearest which the retainer is viewed from in FIG. 35, and shows an actuator on the retainer.

FIG. 37 is a side view of the retainer from FIG. 35 and shows the actuator depressed.

FIG. 38 is an opposite side view of FIG. 36.

FIG. 39 is a top view of the retainer from FIG. 35.

FIG. 40 is a front view of the retainer from FIG. 35, and shows a rotatable collar in a closed position.

FIG. 41 is another front view of the retainer from FIG. 35, and shows the rotatable collar in an open position.

FIG. 42 is a back view of the retainer from FIG. 35.

FIG. 43A is a cross-sectional view of the retainer taken along the line 43-43 of FIG. 39.

FIG. 43B is a cross-sectional view of the retainer taken along the line 43-43 of FIG. 39, and shows the actuator depressed.

FIG. 44 is a perspective view of the harness from FIG. 31.

FIG. 45 is a front view of the securement system from FIG. 31 attached to the intubated patient from FIG. 27, and shows the rotatable collar in an open position.

FIG. 46 is similar to FIG. 45 except that the rotatable collar is in a closed position with the actuator depressed.

FIG. 47 is a side view of the securement system from FIG. 31 attached to the intubated patient from FIG. 27.

FIG. 48 is a back view of the securement system from FIG. 31 attached to the intubated patient from FIG. 27.

FIG. 49 is a perspective view of a securement system in accordance with an embodiment of the present invention and shows a support member, a retainer, connection members, and a strap.

FIG. 50 is a perspective view of the support member from FIG. 49.

FIG. 51 is a top view of the support member from FIG. 50.

FIG. 52 is a front view of the support member from FIG. 50.

FIG. 53 is a side view of the support member from FIG. 50.

FIG. 54 is a back view of the support member from FIG. 50.

FIG. 55 is a top view of a connection member from FIG. 49.

FIG. 56 is a bottom view of the connection member from FIG. 55.

FIG. 57 is a side view of the connection member from FIG. 55.

FIG. 58 is a side view of the securement system from FIG. 49 attached to the intubated patient from FIG. 27.

FIG. 59 is a front view of another embodiment of the securement system from FIG. 49 attached to the intubated patient from FIG. 27 with a plurality of straps.

### DETAILED DESCRIPTION OF CERTAIN EMBODIMENTS

The following description and examples illustrate preferred embodiments of the present securement system disclosed in the context of use with an exemplary endotracheal tube. More specifically, the embodiments relate to a securement system and related techniques that maintain an endotracheal tube in position upon a patient and/or that inhibit migration of the tube within the throat of the patient. The securement system is configured to selectively allow access to the patient's oral cavity. The principles of the present invention, however, are not limited to endotracheal tubes such as those shown. It will be understood by those of skill in the art in view of the present disclosure that the securement system described can be used with other types of medical articles and medical articles of differing sizes, including, but not limited to endotracheal tubes of different design, either with or without tube adaptors, and the like. One skilled in the art may also find additional applications for the devices and systems disclosed herein. Thus, the illustration and description of the securement system in connection with an endotracheal tube is merely exemplary of one possible application of the securement system and technique disclosed.

The securement system retains an endotracheal tube in position on a patient. A support member conforms to the shape of the patient's face. One or more straps can be attached to the support member and each strap can be adjusted both in length and orientation. Alternatively, a head contact member or other type of attachment mechanism or head securement member can be used to attach the securement system to the patient. The straps or head contact member may hold the securement system in place and may additionally keep the support member in contact with the patient's face.

The securement system described herein not only secures an endotracheal tube to a medical patient, it may also inhibit occlusion of the endotracheal tube. The endotracheal tube is deformable and passes between the patient's teeth; thus, there is a risk that the tube may become occluded by a patient's teeth when the patient attempts to bite down. Such occlusion can lead to, for example, hypoxia. The present securement system may be configured to inhibit this occlusion using a blocker to prevent the patient from biting down on the endotracheal tube.

Furthermore, the securement system described herein allows for improved oral care of a patient. To facilitate improved oral care, the system permits access to the patient's oral cavity by a medical practitioner. The medical practitioner can selectively locate the endotracheal tube at a plurality of lateral locations. By adjusting the location of the endotracheal tube, the medical practitioner can more easily access and see into the patient's oral cavity. In addition, the blocker may be moved with the endotracheal tube, thereby inhibiting occlusion of the endotracheal tube at each of the plurality of positions.

In addition, the securement system allows for improved comfort of an intubated patient. A soft gel is located adjacent to the patient's skin. The durometer of the soft gel is low and the soft gel may comprise a medical grade mineral oil. In combination with the conforming support member, the soft gel decreases irritation of the patient's skin and may aid in securement of the endotracheal tube.

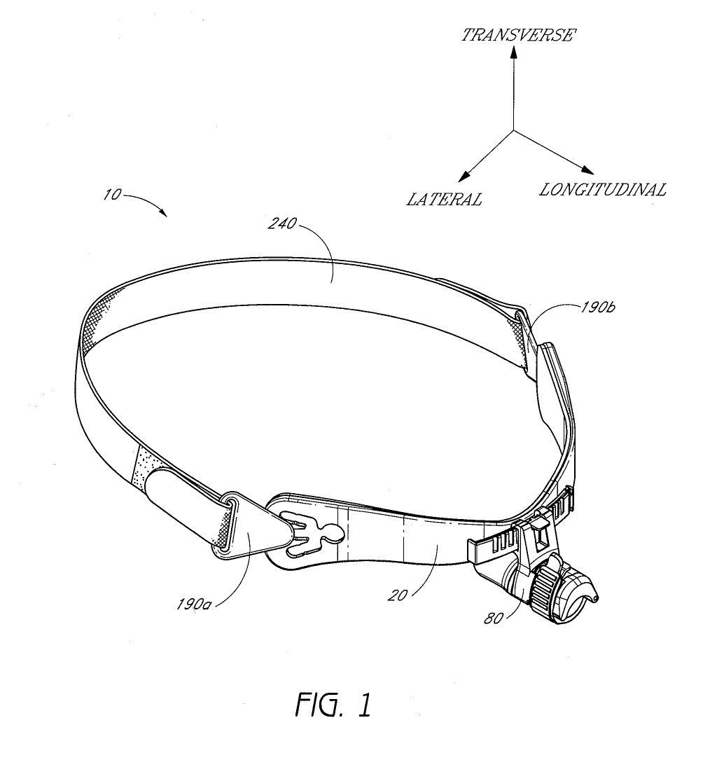

A detailed description of embodiments of a securement system, and its associated method of use, now follows. With reference now to FIG. 1, an embodiment of a securement system 10 includes a support member 20, a retainer 80, connection members 190a and 190b, and a strap 240. The securement system 10 is configured to attach to a patient and secure an endotracheal tube to the patient. The support member 20 is configured to be secured to the patient and inhibit further movement of the support member 20 into the patient's mouth once the support member 20 is secured. The retainer 80 is configured to receive a portion of the endotracheal tube such that the tube is aligned with the patient's mouth. The connection members 190a and 190b are configured to attach the strap 240 to the support member 20. The strap 240 is configured to secure the support member 20 to the patient's face.

To assist in the description of the components of embodiments of the securement system, the following coordinate terms are used, consistent with the coordinate axes illustrated in FIG. 1. A “longitudinal axis” is generally parallel to a portion of a medical article retained by the securement system 10. A “lateral axis” is normal to the longitudinal axis and is generally tangential to the curve of the support member 20 at the location of the retainer 80. When the securement system 10 is secured to the patient, the lateral axis will be generally parallel to a width of the patient's face (i.e. from cheek to cheek).

A “transverse axis” extends normal to both the longitudinal and lateral axes. When the securement system 10 is secured to the patient, the transverse axis will be generally parallel to a length of the patient's face (i.e. from chin to forehead). In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. Also, the terms “proximal” and “distal”, which are used to describe the present securement system 10, are used consistently with the description of the exemplary application. Thus, proximal and distal are used in reference to the center of the patient's body.

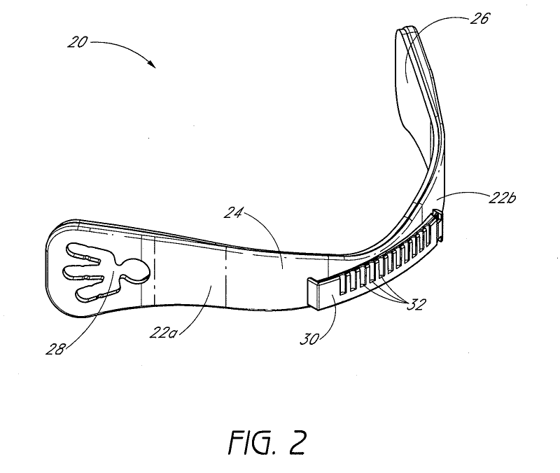

FIGS. 2-7 further illustrate the support member 20 from FIG. 1. As can be seen in a perspective view of the support member 20 in FIG. 2, the support member 20 is comprised of a first side 22a and a second side 22b, which together form a generally curved shape. Configuring the support member 20 as a generally curved shape enhances the fit of the support member 20 with the patient's face. Of course the support member 20 is not limited to a curved shape and may have any other shape.

As can be seen in a top view of the support member 20 in FIG. 3, the first side 22a and second side 22b are illustrated in this embodiment as being substantially symmetrical. The first side 22a and the second side 22b may also be asymmetrical. Such a design may be advantageous, for example, to accommodate different facial shapes or to accommodate medical equipment that may be positioned on a side of the face. Those skilled in the art will appreciate that the first side 22a and the second side 22b may each be shorter or longer, or the proximal ends of the first side 22a and the second side 22b may be spaced closer together or further apart. Such differing configurations of the support member 20 may accommodate differing facial shapes or differing medical applications, for example.

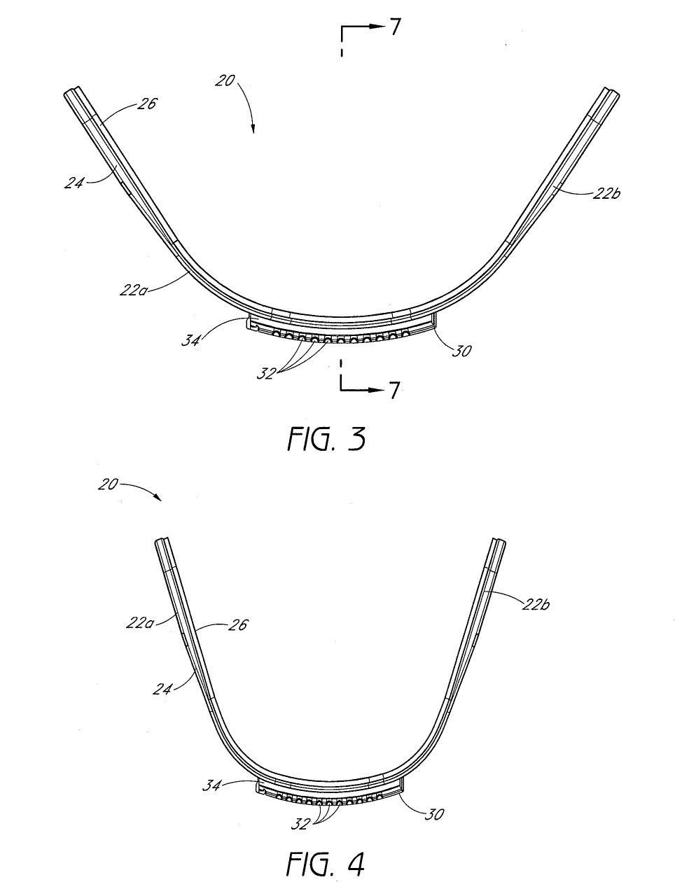

The support member 20 comprises anterior structure 24 comprised of a rigid material. One or more of the first side 22a and the second side 22b of the support member 20, however, are configured to flex and may conform to the shape of a patient's face. For example, FIG. 4 illustrates a top view of the support member 20 and shows the first side 22a and the second side 22b flexed towards each other. Suitable materials for implementing the rigid anterior structure 24 such that the first side 22a and/or the second side 22b may flex include, for example, but without limitation, plastics, polymers or composites such as polypropylene, polyethylene, polycarbonate, polyvinylchloride, acrylonitrile butadiene styrene, nylon, olefin, acrylic, polyester, as well as moldable silicon, thermoplastic urethane, thermoplastic elastomers, thermoset plastics and the like. However, other materials can be utilized.

The support member 20 has a posterior facing surface 26 configured to inhibit movement of the support member 20 into the patient's mouth. In some embodiments, the posterior facing surface 26 comprises a soft gel material. The soft gel material may provide padding for the support member 20 when the support member 20 is placed against the face of a patient, thereby increasing the comfort of the patient. Such soft gel material may also decrease any harmful effects caused by movement of the support member 20 in relation to the patient's face or prolonged contact between the support member 20 and the patient's face.

The soft gel may comprise any number of materials having a low durometer and being suitable for placement against a patient's skin. In some embodiments, the soft gel is impregnated with medical grade mineral oil. The soft gel may also contain moisturizers that treat the patient's skin as the skin is contacted. The soft gel may comprise a material that conforms to the shape of a patient's face, for example by way of heat or pressure or other means, such that a customized fit between the support member 20 and the patient's face is provided.

The soft gel material may be permanently attached to the anterior structure 24. For example, soft gel sheeting may be attached to the anterior structure 24 using an adhesive or other means of attachment. The soft gel material may be overmolded to the anterior structure 24. The soft gel material is configured to bend, flex, or conform with the first side 22a and the second side 22b.

The support member 20 is configured to attach to the connection members 190a and 190b, as illustrated in FIG. 1. In the illustrated embodiment, each of the first side 22a and the second side 22b define at least one recess 28. At least a portion of the connection member 190a or 190b may be inserted into the recess 28 and thereby attach to the support member 20.

As can be seen in a side view of the support member 20 in FIG. 6, the recess 28 is illustrated as having a circular recess 28a connected to three finger-shaped recesses 28b, 28c, and 28d. A protrusion on the connection member 190a or 190b may be inserted into the circular recess 28a and then translated along one of the finger-shaped recesses 28b-28d, as will be describe in more detail below.

As can be seen in a cross-section view of the support member 20 in FIG. 7, the soft gel material is disposed over the posterior side of the recess 28. Thus, when a portion of the connection member 190a or 190b is inserted into the recess 28, the portion will not contact the skin of the patient. In addition, the soft gel material may aid in attaching the connection member 190a or 190b to the support member 20 by placing forcing the portion of the connection member 190a or 190b against the anterior structure 24.

The support member 20 is not limited to the illustrated means for allowing attachment of the connection members 190a and 190b to the support member 20. For example, the recess 28 may be shaped different than illustrated. Each side of the support member 20 may define a recess similar to or different from a recess defined on the other side of the support member 20. Each side may contain a varying number of recesses. In some embodiments, one or both sides omit the recess 28, for example when a strap is substantially permanently connected to the support member 20.

Alternatively, the support member 20 may comprise one or more protrusions for attachment to the connection members 190a or 190b, or the support member 20 may comprise a plurality of recesses and protrusions. In addition, the soft gel material may or may not be disposed over a portion of the anterior side of any recesses in the support member 20. Also, the connection member 190a or 190b may be substantially permanently attached to the support member 20, or the support member 20 may be configured to directly attach to a head securement member or may be substantially permanently attached to a head securement member.

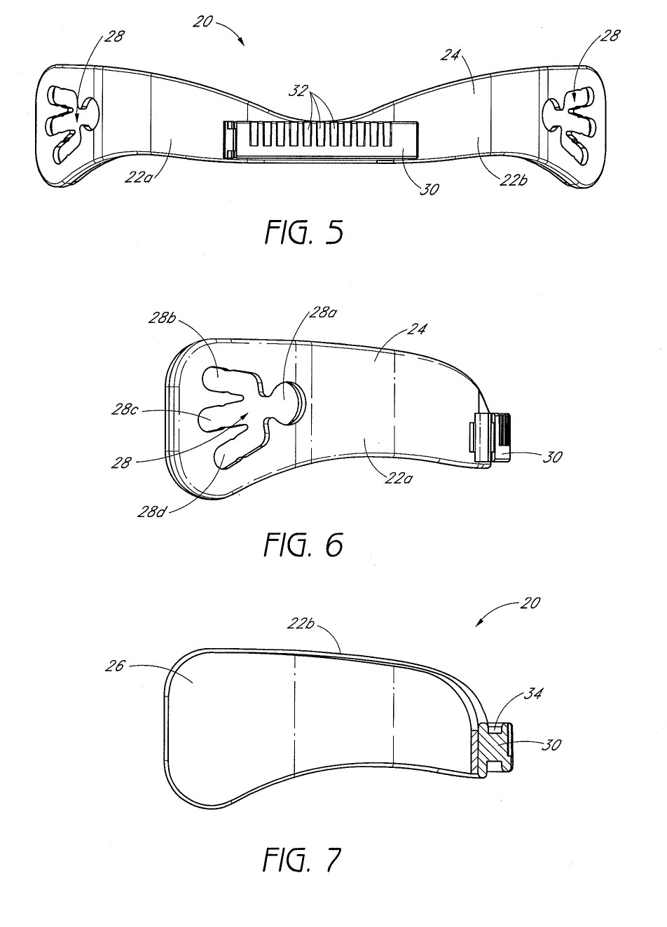

As can be seen in a front view of the support member 20 in FIG. 5, the support member 20 has a track 30. The track 30 is configured to allow attachment of the retainer 80 to the support member 20. In addition, the track 30 allows translation of the retainer 80 without necessitating disconnection of the retainer 80 from the support member 20.

The track 30 has a plurality of interstices 32 that define index locations at which the retainer 80 can be secured. As can be seen in the top view of the support member 20 in FIG. 3, the interstices 32 are located distally of one or more grooves 34. In the illustrated embodiment, the support member 20 includes two parallel grooves 34 on opposite transverse sides of the track 30. The one or more grooves 34 accept portions of the retainer 80. When attached via the groove 34, the retainer may translate along the groove 34 and from interstice to interstice without disconnecting from the support member 20. Of course, either or both of the interstices 32 or the groove 34 may be omitted from the securement system 10.

In the illustrated embodiment, the track is connected to the anterior structure 24 at a location that is substantially centered on the support member 20. The track may be connected using an adhesive, threaded means, or self-locking means, among other attachment means. In other embodiments, the track may be integral to the anterior surface 24. In some embodiments, the track is disposed at another position along the support member 20.

In the illustrated embodiment, the track 30 is configured to allow movement of the retainer 80 in a plane defined by the lateral and longitudinal directions. In other embodiments, the track 30 may be configured to allow movement of the retainer 80 in other directions, such as in a transverse direction. The track 30 may be angled or curved. For example, the track 30 may be curved along a curve of the anterior surface 24, curved in a direction opposed to a curve of the anterior surface 24, or portion of the track 30 may be angled or curved relative to other portions of the track 30. A portion or the entire track 30 may also be substantially linear.

In the illustrated embodiment, the interstices 32 are arranged along the transverse axis. The interstices 32 may, however, be disposed in any direction. In some embodiments, the interstices 32 are angled in order to provide a more secure connection with the retainer 80 or in order to bias the retainer 80 in a certain direction. Of course, each of the interstices 32 need not be disposed in the same direction or have similar shapes.

The anterior structure 24 and the track 30 may be integrally formed. Such an integrally formed structure may be manufactured by any number of processes known in the art, such as by a molding process for example. In other embodiments, any components of the support member 20 may be separately formed and attached together. The track 30 may comprise any of the materials used to manufacture the anterior structure 24.

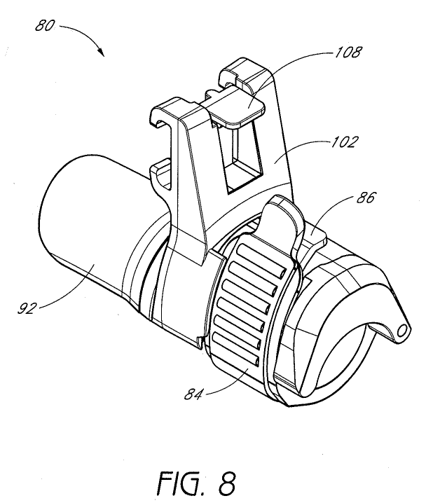

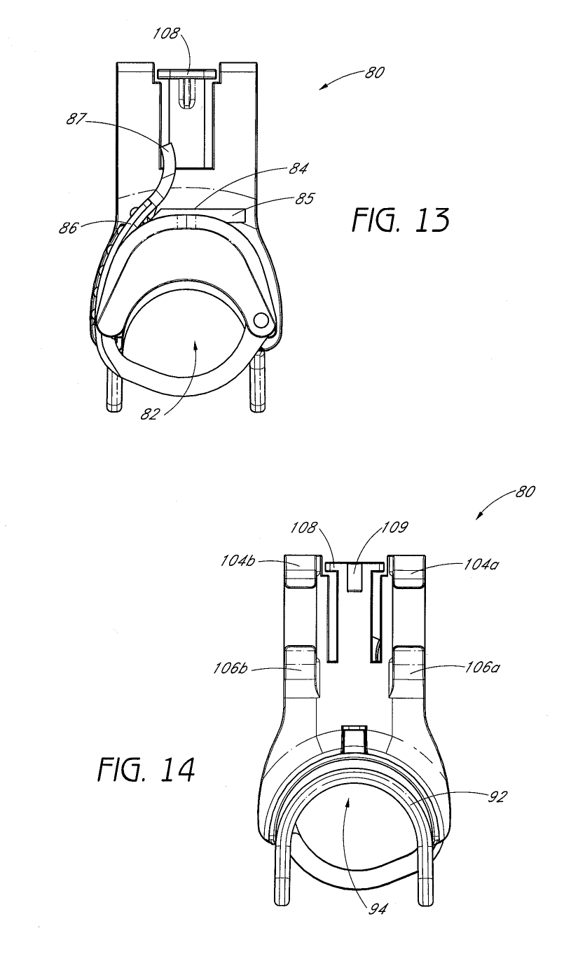

FIGS. 8-15 further illustrate the retainer 80 from FIG. 1. The retainer 80 is configured to align an endotracheal tube with a patient's mouth by attaching to the support member 20. As can be seen in a front view of the retainer 80 in FIG. 13, the retainer 80 forms at least a partial channel 82 that receives at least a portion of an endotracheal tube. The retainer 80 substantially inhibits the endotracheal tube from moving in at least a longitudinal direction when the endotracheal tube is placed in the retainer 80 and the retainer 80 is closed around the endotracheal tube. The retainer 30 preferably further inhibits movement of the endotracheal tube in lateral and transverse directions.

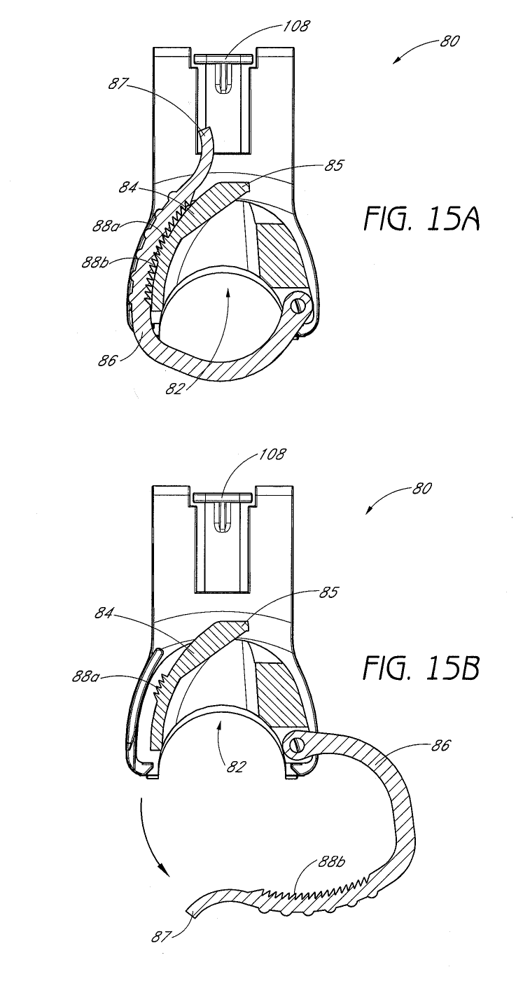

The retainer 80 releasably secures the endotracheal tube. An attachment member 84 and a movable member 86 are configured to form the channel 82 when in a closed position. When in the closed position, a plurality of splines 88a on the attachment member 84 engage with a plurality of splines 88b on the movable member 86, as can be seen in a cross-sectional view of the retainer 80 in FIG. 15A. Those of skill in the art will understand that other means of attachment may be used to secure the movable member 86 to the attachment member 84. For example, complimentary hook and loop fasteners, snaps, or adhesives, among other means, may be used to secure the movable member 86 to the attachment member 84. A front view of the retainer 80 in a closed position is illustrated in FIG. 13.

As most clearly shown in FIGS. 15A and 15B, to disengage the movable member 86 from the attachment member 84, a medical practitioner applies pressure towards the channel 82 in an area 85 of the attachment member 84. Alternatively, the medical practitioner may apply pressure away from the channel 82 to an area 87 of the movable member 86. After the movable member 86 disengages from the attachment member 84, the retainer 80 will be in an open position and may appear as illustrated in a cross-sectional view of the retainer 80 in FIG. 15B, or as illustrated in a side view of the retainer 80 in FIG. 11.

When the movable member 86 is disengaged from the attachment member 84, a medical practitioner may introduce a portion of an endotracheal tube into the channel 82. Thereafter, the medical practitioner may press the moveable member 86 towards the channel 82 and apply sufficient pressure to the movable member 86 to cause the movable member 86 to engage the attachment member 84. Thus, the retainer 80 will enclose the portion of the tube when the movable member 86 and the attachment member 84 are engaged, thereby surrounding the circumference of the tube. An outside surface of the movable member 86 may be textured, such as with the raised bumps seen in a side view of the retainer 80 in FIG. 10, to increase the ease with which the medical practitioner may press the movable member 86 towards the attachment member 84.

The retainer 80 is configured to accept medical articles of varying shapes and sizes. The splines 88b on the movable member 86 can engage the splines 88a on the attachment member 84 at a plurality of positions. Thus, the size and shape of the channel 82 defined by the attachment member 84 and the movable member 86 can vary. In one embodiment, the retainer is configured to retain endotracheal tubes from a size 5 to a size 10. In some embodiments, the movable member 86 may be omitted.

In the illustrated embodiment, an enclosed area formed when the movable member 86 and the attachment member 84 engage can vary from being roughly circular to being substantially oval or elliptical. In this embodiment, the retainer 80 can securely surround an endotracheal tube and lock the tube into position when the attachment member 84 and the movable member 86 are engaged. In other embodiments, the enclosed area may be rectangular or of another shape. Such configurations may substantially prevent lateral, transverse, and/or longitudinal motion of an endotracheal tube when the tube is enclosed by the retainer 80.

The retainer 80 may include a bite block 92, as can be seen in a perspective view of the retainer 80 in FIG. 8. The bite block 92 inhibits a patient's teeth from contacting the endotracheal tube in the patient's mouth. Thus, if the patient were to bite down, the bite block 92 would diminish the possibility of the tube being punctured or occluded.

The bite block 92 may include at least a partial channel. As can be seen in a back view of the retainer 80 in FIG. 14, the illustrated embodiment of the bite block 92 includes a channel 94. The channel 94 is preferably coaxially aligned with the channel 82 through the retainer 80. The channel 94 accepts a portion of an endotracheal tube. The channel 94 can be shaped in any way that allows the endotracheal tube to be placed at least partially therein. In the illustrated embodiment, the channel 94 is generally semicircular, but other shapes may be selected.

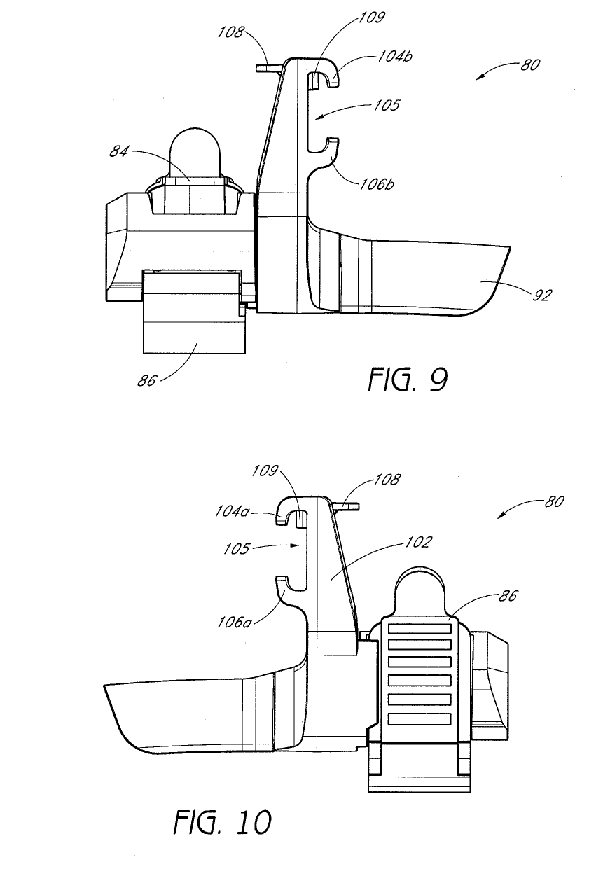

The retainer 80 includes an arm 102. The arm 102 is configured to attach to the track 30. As can be seen in a side view of the retainer 80 in FIG. 9, the retainer 80 includes upper and lower protrusions 104 and 106. A receiving space 105 defined between the upper and lower protrusions 104 and 106 is configured to accept at least a portion of the track 30.

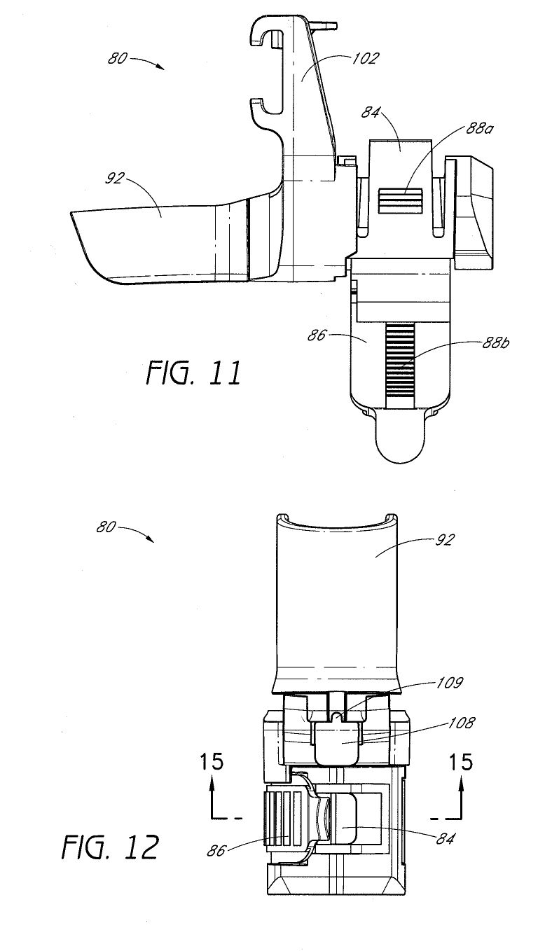

As can be seen in the back view of the retainer 80, the upper and lower protrusions 104 and 106 are comprised of left protrusions 104a and 106a, respectively, and right protrusions 104b and 106b, respectively. The left protrusions 104a and 106a are separated from the left protrusions 104b and 106b buy an actuator 108 attached to a center protrusion 109, as can be seen in a top view of the retainer 80 in FIG. 12.

The upper protrusions 104 are configured to allow the retainer 80 to be supported by the track 30. In the illustrated embodiment, the upper protrusions 104 are configured in a hook shape such that they can be placed within the upper groove 34. After placement in the groove 34, the upper protrusions 104 may allow the retainer 80 to slide along the track 30 without needing to detach the retainer 80 from the support member 20. The groove 34 may contain sidewalls to inhibit the retainer 80 from sliding off of the track 30.

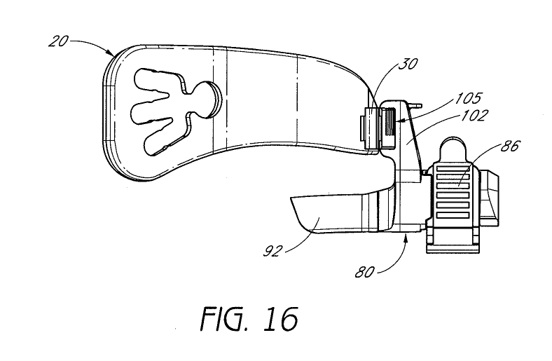

In the illustrated embodiment, the bottom protrusions 106 are also configured in a hook shape. Such configuration of the bottom protrusions 106 may allow the bottom protrusions 106 to be placed within the groove on the bottom of the track. This bottom groove may be configured similar to the upper groove 34. In this configuration, the retainer 80 is capable of partially enclosing a portion of the track 30 within the receiving space 105 and increasing the security with which the retainer 80 is attached to the support member 20. This can be most easily seen in a side view of the retainer 80 attached to the support member 20, illustrated in FIG. 16. Those of skill in the art will recognize that the hooked portions of the top protrusions 104 and the bottom protrusions 106 inhibit the retainer 80 from being pulled from the support member 20 in at least a lateral direction.

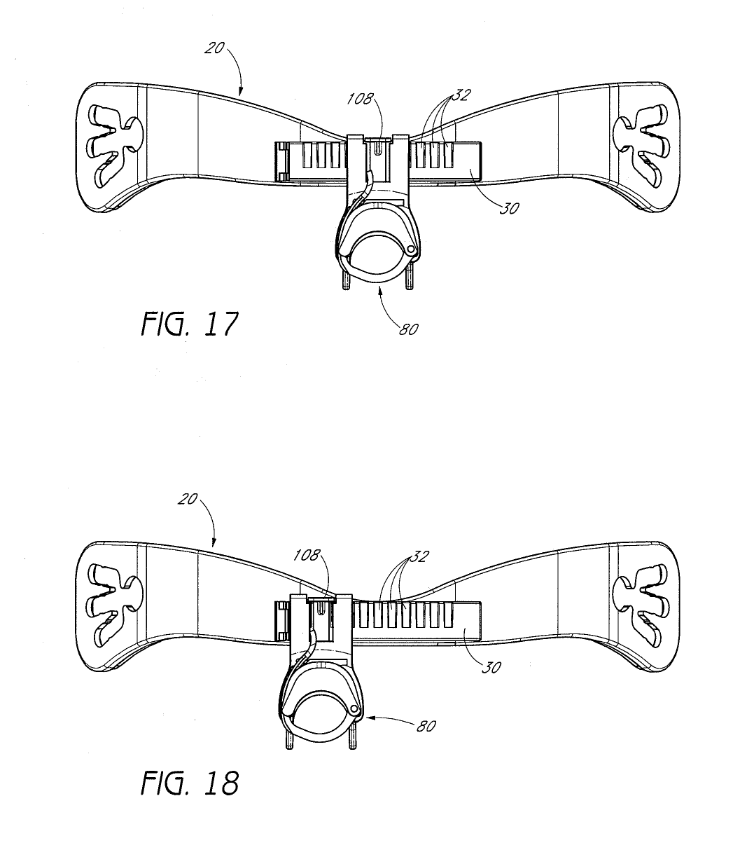

The actuator 108 and the center protrusion 109 are configured to secure the retainer 80 at a selected index location along the track 30. The center protrusion 109 is configured for placement in a selected one of the interstices 32. When the center protrusion 109 is placed in one of the interstices 32, the retainer 80 will be inhibited from traversing the track 30. In combination with the upper protrusions 104 and the lower protrusions 106, the center protrusion may lock the retainer 80 in place at a selected location along the track 30, as is illustrated in FIG. 17.

A medical practitioner may apply pressure to the actuator 108 to cause the center protrusion 109 to lift or bend out of one of the interstices 32. Thereafter, the medical practitioner may move the retainer 80 to another location along the track 30, as is illustrated in the FIG. 18. If the center protrusion 109 is aligned with another of the interstices 32 at this new location, then the center protrusion 109 will enter the interstice and secure the retainer 80 in that location. Such relocation may be performed as often and as many times as necessary without substantially damaging either the support member 20 or the retainer 80.

Those of skill in the art will recognize that in the illustrated embodiment, the bite block 92 will be relocated with the retainer 80 when a medical practitioner translates the retainer 80. Thus, the bite block 92 will continue to protect an endotracheal tube placed at least partially inside, regardless of the positioning of the retainer 80.

Those of skill in the art will understand that one or more of the upper protrusions 104, lower protrusions 106, actuator 108, and center protrusion 109 may be omitted from the retainer. In some embodiments, the track 30 includes a plurality of protrusions and the retainer 80 includes at least one recess configured to accept any of the protrusions. In addition, each of the upper protrusions 104, lower protrusions 106, actuator 108, and center protrusion 109 may be configured in a different shape or form than is illustrated. For example, the lower protrusions 106 may be substantially linear such that they extend in solely a longitudinal direction from the retainer 80, or the lower protrusions 106 may be omitted altogether.

The retainer 80 may be integrally formed. Such an integrally formed structure may be manufactured by any number of processes known in the art, such as by a molding process for example. In other embodiments, components of the retainer 80 may be separately formed and attached together. The retainer 80 may comprise any of the materials used to manufacture the support member 20.

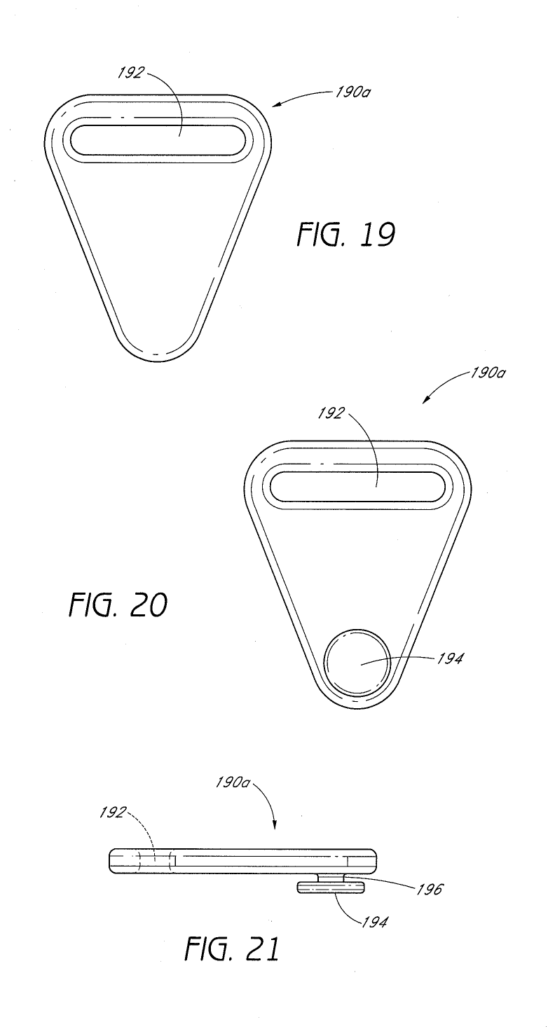

FIGS. 19-21 further illustrate the connection member 190a from FIG. 1. The connection member 190a is configured to attach the strap 240 to the support member 20. The connection member 190a defines an opening 192 therethrough, as can be seen in a top view and a bottom view of the connection member 190a in FIG. 19 and FIG. 20, respectively. The opening 192 accepts a portion of the strap 240, illustrated in FIG. 1. The size and shape of the opening 192 roughly correspond to a cross-section of the strap 240. Of course, the opening 192 can be larger than the cross-section of the strap 240. Matching a cross-section of the strap 240 to the size and shape of the opening 192, however, minimizes longitudinal and/or transverse movement of the strap 240 when placed through the connection member 190a.

The connection member 190a includes a protrusion. The protrusion may be inserted into the recess 28 in the support member 20 to attach the connection member 190a to the support member 20. In the illustrated embodiment, the protrusion includes a head 194 and a neck 196 that is narrower than the head 194. Thus, a portion of the support member 20 can be captured between the head 194 and another portion of the connection member 190a adjacent to the neck 196. Such captured portion of the support member 20 will inhibit disconnection of the connection member 190a from the support member 20.

In the illustrated embodiment, the neck 196 is substantially circular. When a portion of the support member 20 is captured adjacent to the neck 196, the connection member 190a will be able to rotate or pivot about the neck 196. This rotation allows a strap attached to the connection member to be placed in a variety of orientations about the head of a patient, and facilitates the use of multiple straps in the securement system 20. The neck, however, may be configured as a different shape or size than is illustrated. Some shapes, such as a rectangle, may be selected to inhibit rotation so that a strap connected to the connection member 190a can be disposed in a previously determined orientation.

The head 194 may be of any shape that can be inserted into the recess 28 of the support member 20. In the illustrated embodiment, the head is substantially circular and is shaped similar to the circular recess 28a. Configuring the head 194 and the recess 28a as similar shapes decreases the chance of the connection member 190a becoming unattached from the support member 20 unintentionally after insertion of the head 194 into the recess 28.

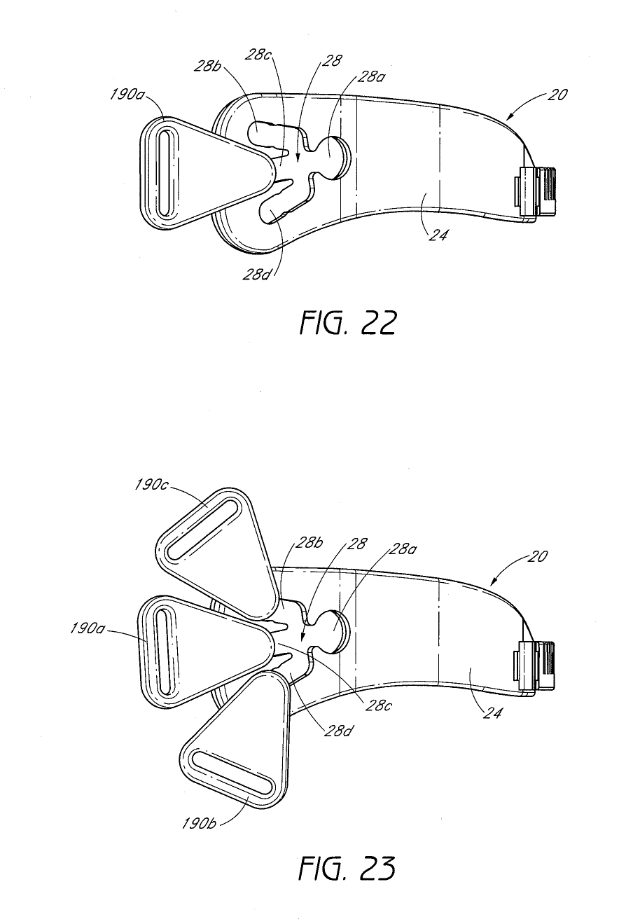

To attach the connection member 190a to the support member 20, a medical practitioner aligns the head 194 with the circular recess 28. The medical practitioner then causes the head 194 to enter the recess and pass beyond the anterior structure 124 of the support member 20. After this is completed, the neck 196 will be located adjacent to the anterior structure 24. The medical practitioner can then cause the connection member 190a to slide into one of the finger-shaped recesses 28b-28d, as illustrated in FIG. 22.

In the illustrated embodiment, the finger-shaped recesses 28b-28d are narrower than the circular recess 28a. Thus, the head 194 will be captured on a posterior side of the anterior surface 24 and the head 194 can apply pressure to the support member 20, such as to pull the support member 20 against the face of a patient. The soft gel of the support member 20 is illustrated as being able to pressure the head 194 against the anterior structure 24 and thereby increase the security of the attachment of the connection member 190a to the support member 20.

In the illustrated embodiment, the finger-shaped recesses 28b-28d are similarly shaped and the connection member 190a may be placed in any one of the finger-shaped recesses 28b-28d by the medical practitioner. In other embodiments, the connection member 190a or the recess 28 is shaped so that the connection member 190a may be inserted at only one location or guided only along one path.

When pulled to the end of one of the finger-shaped recesses 28b-28d, the connection member 190a will abut a portion of the anterior structure 24. Thus, when a strap is attached to the connection member 190a and placed around the head of a patient, the connection member 190a will pull the support member 20 towards the patient.

The connection member 190a is shown as being located in finger-shaped recess 28c in FIG. 22. The medical practitioner may, however, guide the connection member 190a out of the finger-shaped recess 28c and into another finger-shaped recess 28b or 28d. Each finger-shaped recess 28b may therefore define one or more attachment points at which the connection member 190a can attach to the support member 20. Alternatively, the connection member 190a may be guided back to the circular recess 28a and removed from the support member 20. Attachment, relocation, and/or removal may be performed as often and as many times as necessary without substantially damaging either the support member 20 or the connection member 190a.

Those of skill in the art will appreciate that the connection member 190a may be rotated while attached to the support member 20. Thus, a strap attached to the connection member 190a can be positioned into any angle of orientation about an axis of the neck 196. As discussed above, the connection member 190a may be configured to instead inhibit such rotation or to be placed at a number of predetermined rotations.

The connection member 190a may be configured as any shape. In the illustrated embodiment, the connection member 190a is substantially triangular. Such triangular shape allows for multiple connection members to be attached to a single side of the support member 20, as shown in FIG. 23. The triangular shape also provides enough surface area for an opening and a protrusion to be formed in the connection member, while simultaneously substantially minimizing the amount of material required to form the connection member.

As described above, FIG. 23 illustrates that a connection member in addition or in place of the connection member 190a may be attached to the support member 20. The additional connection member or members may be configured substantially similar to the connection member 190a, as are connection members 190b and 190c in the illustrated embodiment, or the additional connection member may be configured different than the connection member 190a. For example, one or more of the connection members may have a rectangular shape to increase the ease with which the medical practitioner can grasp it.

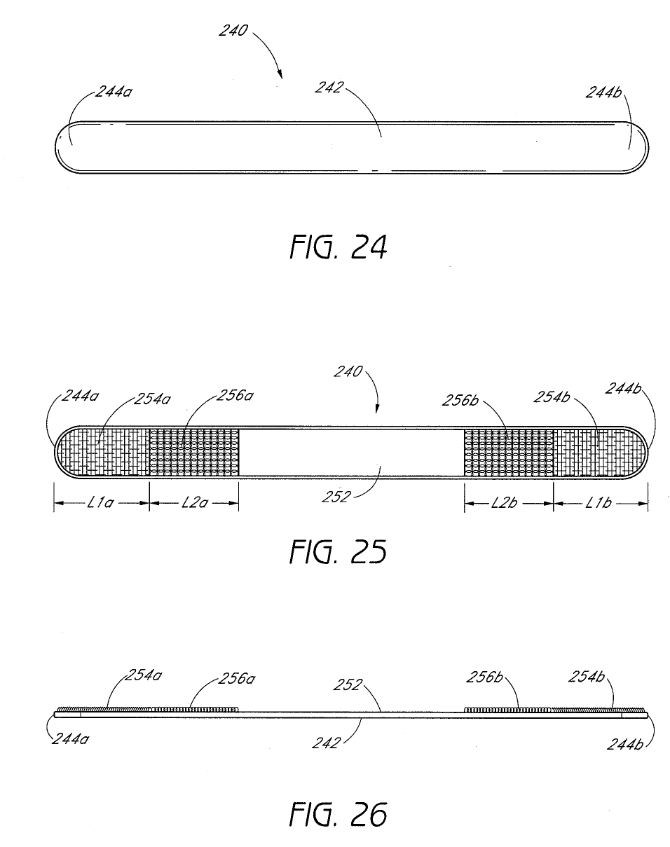

FIGS. 24-26 further illustrate the strap 240 from FIG. 1. As illustrated in a top view of the strap 240 in FIG. 24, the strap is configured such that at least the ends 244a and 244b of the strap 240 may be passed through the opening 192 of the connection member 190a, illustrated in FIG. 19. In the illustrated embodiment, the strap 240 is further configured to be placed around the back of a patient's head and/or neck. Thus, a length of the strap 240 allows the strap 240 to pass through the opening 192 of the connection member 190a, travel around the back of a patient's head and/or neck, and pass through an opening in the connection member 190b, as illustrated in FIG. 1. In addition, an upper surface 242 of the strap 240 is configured to comfortably be placed in contact with a patient's hair and/or skin.

The shape and construction of the strap 240 may otherwise be varied. Any number of shapes or designs of the strap 240 are possible and within the scope of this description. For example, although the strap 240 is illustrated as being substantially uniform in width, the strap 240 may be shaped so as to be wider at its middle to provide a greater contact area with the head and/or neck of the patient. Although the strap 240 is illustrated as having rounded ends 244a and 244b, which may ease placing the ends 244a and 244b through the opening 192, other embodiments include a strap with ends that are not rounded or with a single rounded end. One such embodiment is a strap with squared ends, which may facilitate a secure connection when the strap 240 is attached to the connection member 190a and. Although the strap 240 is illustrated as a single piece of material, the strap 240 may also comprise several pieces of material attached together.

Viewed from the bottom, as illustrated in FIG. 25, the strap 240 is comprised of a lower surface 252, hook portions 254a and 254b, and loop portions 256a and 256b. In one embodiment, the hook portions 254a and 254b and the loop portions 256a and 256b are attached onto the lower surface 252, as illustrated in FIG. 25. In another embodiment, the lower surface 252 may only extend to the beginning of the hook portions 254a and 254b or the loop portions 256a and 256b, and the remaining portions may be attached to each other laterally instead of placed on the lower surface 252. Thus, the strap 240 may be comprised of multiple sections or portions attached together. The lower surface 252 may be integral to the upper surface 252, illustrated in FIG. 26, or the lower surface 252 may be separate from and stacked on or laminated to the upper surface 242. In one embodiment, at least a part of the lower surface 252—or similarly the upper surface 242—comprises a stretchable or deformable material, which may increase the ease of securing the strap 240 about the head of the patient and increase the tension in the strap 240 once secured. For example, the lower surface 252 may comprise an elastic material.

The hook portion 254a and the loop portion 256a are sequentially disposed on the end 244a of the strap 240, and the hook portion 254b and the loop portion 256b are sequentially disposed on the end 244b of the strap 240. When the end 244a is placed through the opening 192, for example, the end 244a can be folded back toward the rest of the strap 240 and the hook portion 254a can mate with the loop portion 256a, thereby attaching the strap 240 to the connection member 190a. The hook portion 254b can similarly mate with the loop portion 256b when the end 244b is placed through the opening 192.

Either the hook portion 254a or the loop portion 256a may be located next to the end 244a. FIG. 25 illustrates the hook portion 254a as being located next to the end 244a, but the placement of the hook portion 244a and the loop portion 256a could be reversed, with the loop portion 256a being located next to the end 244a. The hook portion 254a and the loop portion 256a may directly abut each other or there may be a distance between the portions.

The hook portion 254a and/or the loop portion 256a can span the entire width of the strap 240. In another embodiment, the hook portion 254a and/or the loop portion 256a may be configured as various shapes or may only partially cover the width of the strap 240.

The hook portions 254a and the loop portion 256a have lengths L1a and L2a, respectively, such that when the strap 240 is attached to the connection member 190a and placed around the patient's head, at least a portion of the hook portion 254a can be passed through the opening 192 and folded back to contact at least a portion of the loop portion 256a. The hook portion 254b and the loop portion 256b may be configured similar to the hook portion 254a and the loop portion 256a with respect to each other and with respect to the end 244b.

Of course, the strap 240 is not limited to attaching to the support member 20 as described above. For example, connection members may be omitted from the securement system 10. In some such embodiments, the strap 240 is configured to attach directly to the support member 20. In this embodiment, the strap 240 may be releasably or substantially permanently attached to the support member 20. Alternatively, the securement system 10 may be configured to utilize a support member with strap that is both directly connected to the support member 20 and that also attaches to a connection member for connecting to the support member 20.

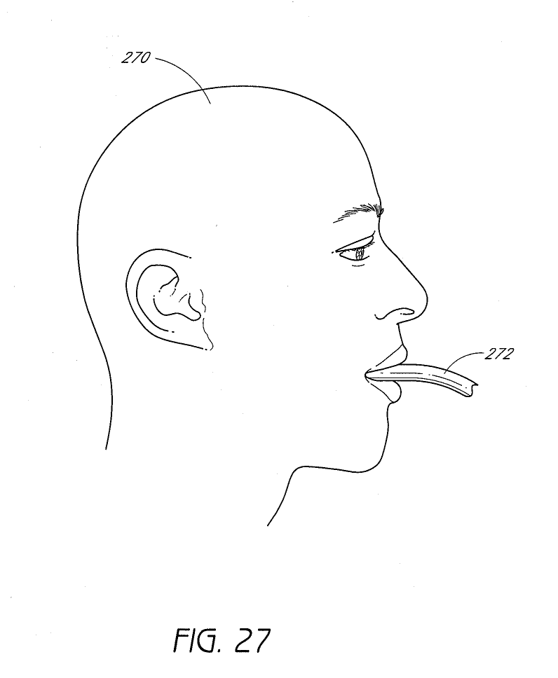

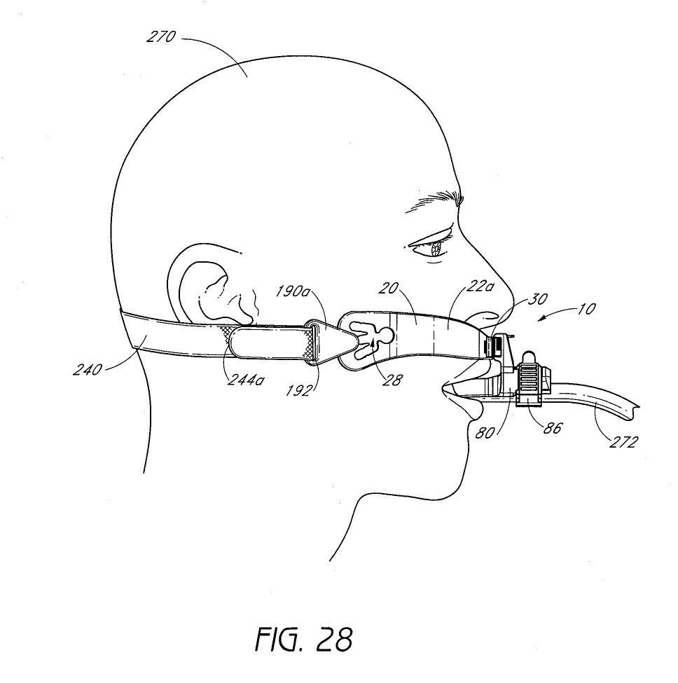

A side view of a patient 270 intubated with an endotracheal tube 272 is illustrated in FIG. 27. A medical practitioner can secure the endotracheal tube 272 using the securement system 10 illustrated in FIG. 1. A side view of the securement system 10 secured to the patient 270 is illustrated in FIG. 28. The support member 20 rests against the face of the patient 270, while the retainer 30 receives a portion of the endotracheal tube 272 and aligns the endotracheal tube 272 with the mouth of the patient 270.

The securement system 10 is secured to the patient 270 by the strap 240. A medical practitioner may place the end 244a through the opening 192 of the connection member 190a, for example, positioning the lower surface 252 of the strap 240 to face away from the support member 20. The medical practitioner can fold the end 244a back towards the strap 240, causing the hook portion 254a to mate with the loop portion 256a. The medical practitioner may attach the connection member 190a to the support member 20, as described above, before or after attaching the strap 240 to the connection member 190a.

The medical practitioner can draw the strap 240 around the back of the head or neck of the patient 270 and then place the end 244b through an opening in the connection member 190b, for example, if the connection member 190b is already attached to the support member 20. The end 244b can then be folded back towards the strap 240, causing the hook portion 254b to mate with the loop portion 256b, thereby attaching the strap 240 to the connection member 190b and the support member 240, and therefore the patient 270.

If the connection member 190b is not attached to the support member 20 already, the strap 240 may be attached to the connection member 190b first. Then the connection member 190b and the strap 240 may be drawn around the back of the head or neck of the patient 270 and the connection member 190b attached to the support member. Thus, the securement system 10 will be secured to the patient 270.

Those of skill in the art will recognize that the strap 240 may be attached to both the connection member 190 and the connection member 190b before either connection member is attached to the support member 20. In this situation, the medical provider can attach one of the connection members 190a or 190b to the support member 20, and then draw the other connection member around the back of the head or neck of the patient 270. The other connection member may then be attached to the support member 20.

Alternatively, the strap 240 and connection members 190a and 190b all be attached to the support member 20 before the securement system 10 is placed on the patient. In such situation, the medical practitioner may start by placing the securement system 10 over the top of the head of the patient 270. Then, the medical practitioner may pull the support member 20 down to the proper position on the face of the patient 270, while simultaneously pulling or stretching the strap 240 around the head and/or neck of the patient 270.

When both ends of the strap 240 are not permanently attached to either of the connection members 190a and 190b or to the support member 20, the medical practitioner may adjust the placement of the securement system 10 or the fit of the securement system 10 against the face of the patient 270 using the hook portions 254a and 254b and the loop portions 256a and 256b. The hook portions 254a and 254b and the loop portions 256a and 256b permit adjustment to the length of the strap 240, such that the posterior facing surface 26 of the support member 20 can be properly tensioned against the face of the patient 270. As discussed above, the soft gel material of the posterior surface may increase the comfort of the patient and may also conform to the patient's face.

When tension is applied to the strap 240, such as by adjusting the length of the strap 240 as described above, the strap 240 will pressure the sides 22a and 22b of the support member 20 towards the face of the patient. This may cause the support member 20 to flex and conform to the shape of the patient's face, thereby providing a more secure and comfortable fit.

The fit and/or placement of the securement system 10 on the patient 270 may also be adjusted by rotating the connection members 190a and 190b to a different orientation so that the strap 240 passes over a different section of the head or neck of the patient. Also, the connection members 190a and 190b can be moved into any portion of the recess 28 on the sides of the support member 20. Thus, the strap 240 can not only be rotated into any orientation, the pivot point for such rotation can be placed at a plurality of locations. In this way, the securement system 10 can be adjusted in a multitude of ways to ensure proper securement.

To add to the above methods of increasing fit, placement, and comfort of the securement system 10, a plurality of strap may be attached to the securement system 10. As described above, a plurality of connection members may be attached to the support member 20. Straps may then connect to a plurality of connection members attached to each side of the support member 20. Those of skill in the art will recognize that the adjustment of straps, rotation of connection members, placement of connection members, and number of connection members and straps can be combined to adjust the securement system 10 to virtually any number of fits and configurations for proper securement to the patient 270.

At any time before, after, or concurrently with attaching the support member 20 to the patient 270 or adjusting the securement system 10 on the patient 270, a medical practitioner can secure the endotracheal tube 272 with the retainer 80. To do so, the medical practitioner places the retainer 80 into an open position, as described above, if the retainer 80 is not already opened. The medical practitioner may then place the retainer 80 over a portion of the endotracheal tube 272 such that the portion is at least partially within the channel 82. Subsequently, the medical practitioner can swing the moveable member 86 around the endotracheal tube 272 and secure the retainer 80 in a closed position, as described above.

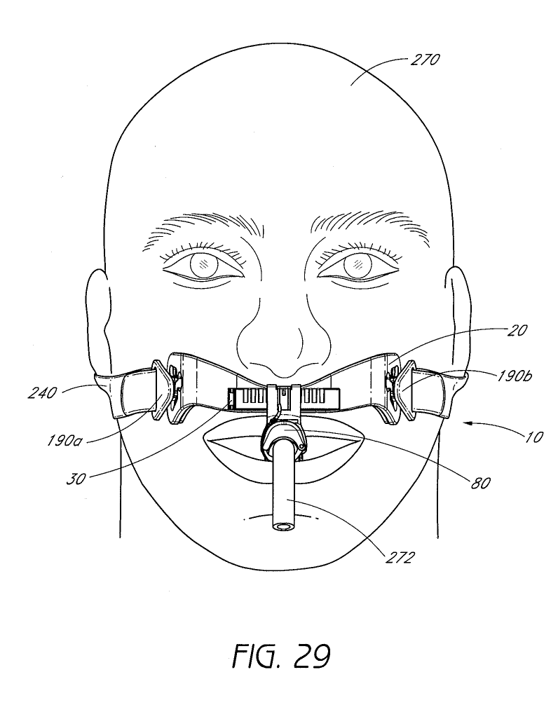

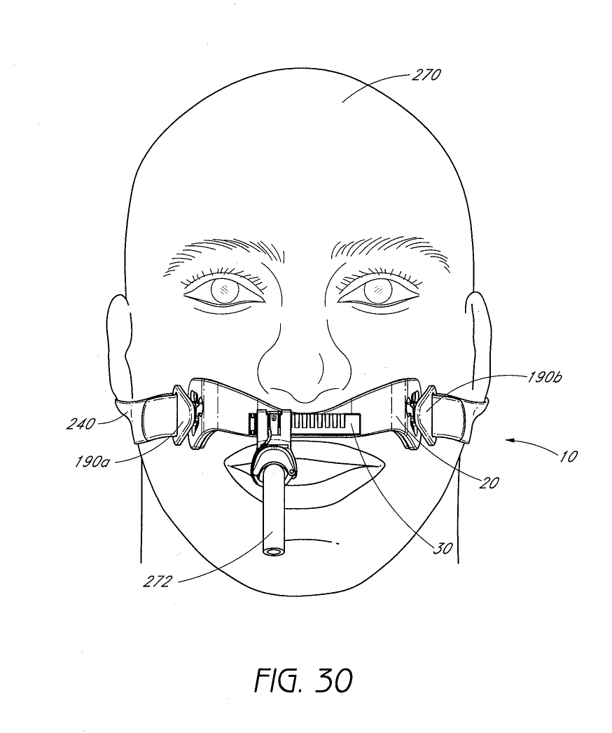

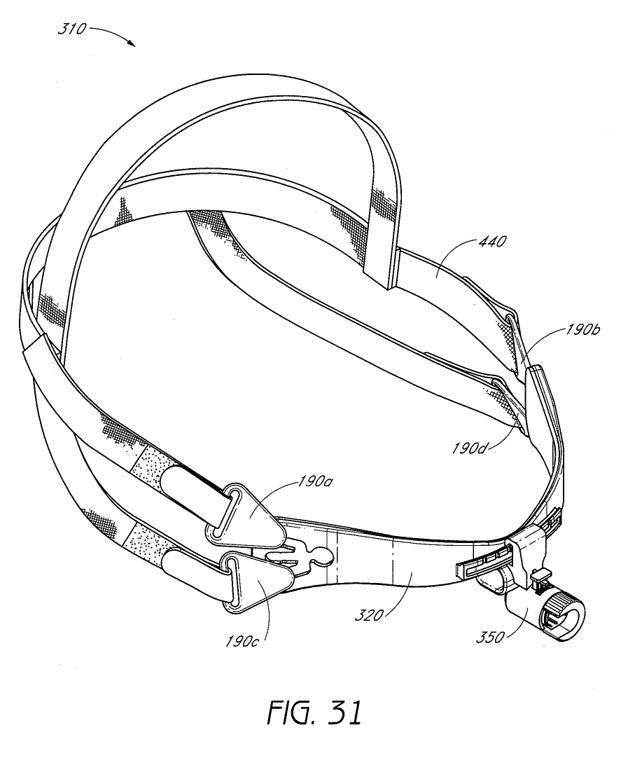

As described above, the position of the retainer 80 on the track 30 may be selected by the medical practitioner. Most commonly, an endotracheal tube is secured in the center of the patient's mouth. The endotracheal tube 272 is shown as centered relative to the mouth of the patient 270 in FIG. 29. The retainer 80 and the endotracheal tube 272 may, however, be relocated to a different position along the track 30 by the medical practitioner. The track 30 allows steady and precise relocation of the endotracheal tube 272 such that the patient 270 is not disturbed or harmed.

Relocating the retainer 80 and the endotracheal tube 272 to a side of the mouth of the patient 270, as illustrated in FIG. 30, allows the medical practitioner to have access to the patient's mouth. Such access is beneficial to the patient 272, for example to allow the medical practitioner to cleanse the mouth of the patient 272. By using the interstices 32 and the center protrusion 109, the medical practitioner can lock the endotracheal tube 272 into position at the side of the mouth of the patient 272. Thus, access to the oral cavity of the patient 272 will be maintained, even if the medical practitioner must use both hands to attend to other matters.

Those of skill in the art will appreciate that the endotracheal tube 272 can be easily and securely repositioned relative to the mouth of the patient 272. This repositioning can be executed without removing or further adjusting the securement system 10 relative to the patient 270. Similarly, adjustment of the fit or positioning of the securement system 10 may be achieved without removing the endotracheal tube 272 from the retainer 80 and/or without removing the securement system 10 from the patient 270.

Those of skill in the art will also appreciate that any other sort of head securement member, in addition to or in place of the strap 240, may be used to attach the securement system 10 to the patient 270. For example, a head contact member in the form of a curved plate or other shape may be connected to the support member 20 and placed behind the head or neck of the patient 270. Alternatively, a plurality of individual or interconnected straps or bands may be used. The head securement member need not wrap entirely around the head or neck of the patient 270. For example, the head contact member may wrap around the ears of the patient 270.

The securement system 10 can be provided as a kit that includes the support member 20, the retainer 80, the connection members 190a and 190b, and the strap 240, all preassembled. In this configuration, the medical practitioner is only required to open the kit, detach one end of the strap 240 from one of the connection members 190a or 190b, attach the securement system 10 to the patient by drawing the strap 240 around the head or neck of the patient and reattaching the end of the strap, and secure the endotracheal tube using the retainer 80. To reposition the endotracheal tube, the actuator 108 is depressed and the retainer 80 is translated. The endotracheal tube may be repositioned at any location relative to the mouth of the patient.

As described above, the support member 20, the retainer 80, the connection members 190a and 190b, and the strap 240 may be packaged and delivered to the medical practitioner in an assembled state. Of course the medical practitioner could assemble one or more of the components of the securement system 10. For example, the connection members 190a and 190b and/or the strap 240 could be delivered in an unassembled state to the medical practitioner. The medical practitioner could then assemble the strap 240 to the connection members 190a and 190b and/or assemble the connection members 190a and 190b to the support member 20.

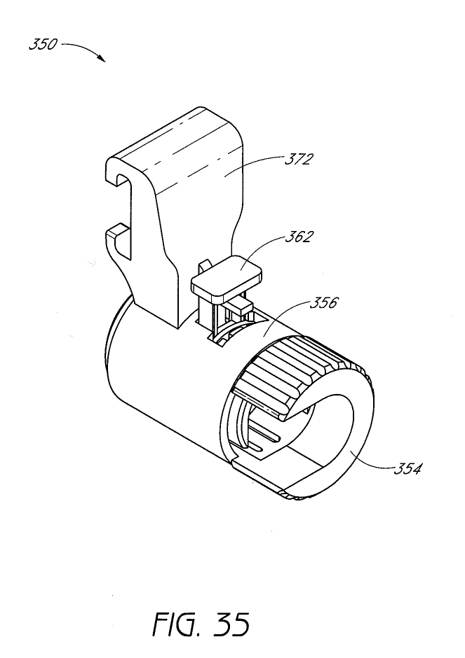

The kit can include additional components for use with the securement system 10. For example, the kit may include additional connection members and/or additional straps. Additional components may be similarly configured or may be configured differently. For example, a head securement member different from a strap could be provided in addition to one or more straps. Also, a different type of retainer, such as described below in reference to FIG. 35, could be provided for use with the securement system 10.

With reference now to FIG. 31, another embodiment of a securement system 310 includes a support member 320, a retainer 350, connection members 190a-190d, and a harness 440. Similar to the securement system 10 illustrated in FIG. 1, the securement system 310 is configured to attach to a patient and secure an endotracheal tube to the patient.

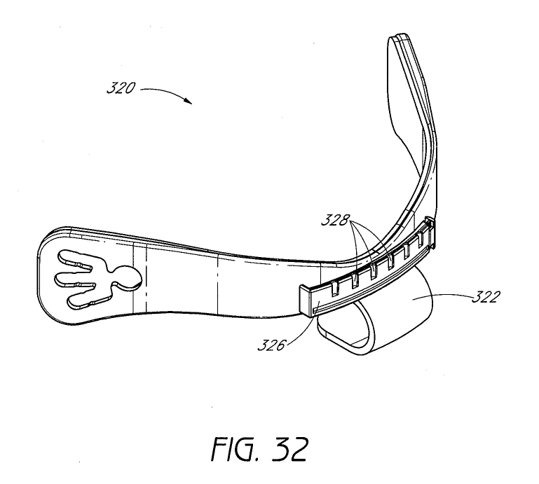

FIGS. 32-34 further illustrate the support member 320 from FIG. 31. The support member 320 is configured to be secured to the patient and inhibit further movement of the support member 320 into the patient's mouth once the support member 320 is secured.

As can be seen in a perspective view of the support member 320 in FIG. 32, the support member 320 includes a bite block 322. The bite block 322 inhibits a patient's teeth from contacting an endotracheal tube in the patient's mouth. The bite block 322 is configured as a curved shape, wherein a curved portion 323 protrudes from the front of the support member 320. Thus, a receiving space 324 is defined in which a patient's upper lip can be positioned when the support member 320 is attached to the patient. This receiving space increases the comfort, of the patient by reducing the chance that the patient's upper lip will rub on the support member 320.

A lower portion 325 of the bite block 322 is connected to the curved portion 323. The lower portion is located beneath the curved portion 323 and more distally. The lower portion 325 is configured to be located beneath a patient's upper teeth when the support member 320 is attached to the patient. If the patient attempts to bite down, the lower portion will inhibit occlusion of an endotracheal tube passing beneath it.

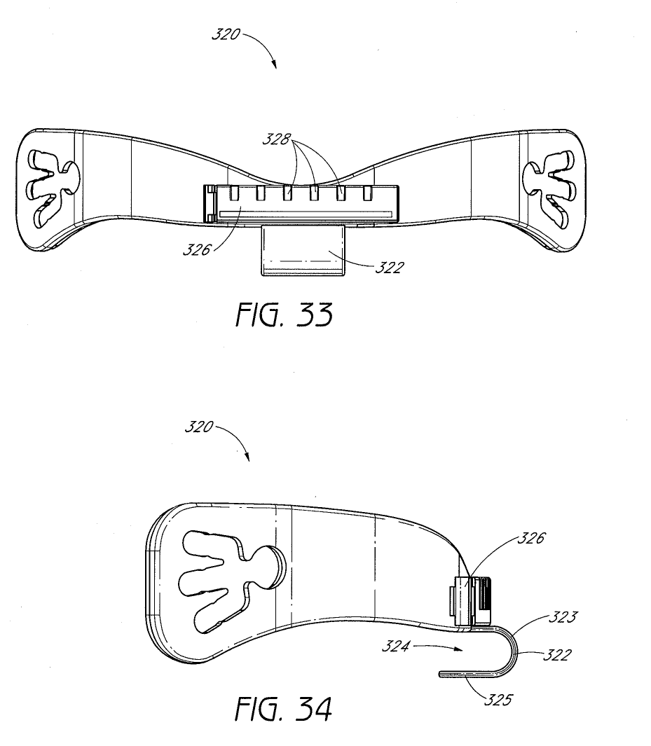

As can be seen in a front view of the support member 320 in FIG. 33, the bite block 322 is illustrated as being substantially laterally centered on the support member 320. This alignment increases the likelihood that the bite block 322 will be contacted instead of an endotracheal tube if the patient bites down. The bite block 322, however, may be offset from center or omitted altogether.

As can also be seen in the front view of the support member 320, the support member 320 includes a track 326. Interstices 328 of the track are spaced farther apart than the interstices 32 of the track 30, illustrated in FIG. 5. Such spacing of the interstices 328 still allows a retainer, such as the retainer 350, to translate along the track 326, but provides fewer locations at which the retainer 350 can be secured.

The support member 320 may otherwise be configured similar to the support member 20 of securement system 10, illustrated in FIG. 2. For example, the support member 320 may be shaped similar to the support member 20. The support member 320 may also have recesses, an anterior structure, and a posterior surface similar to the support structure 20.

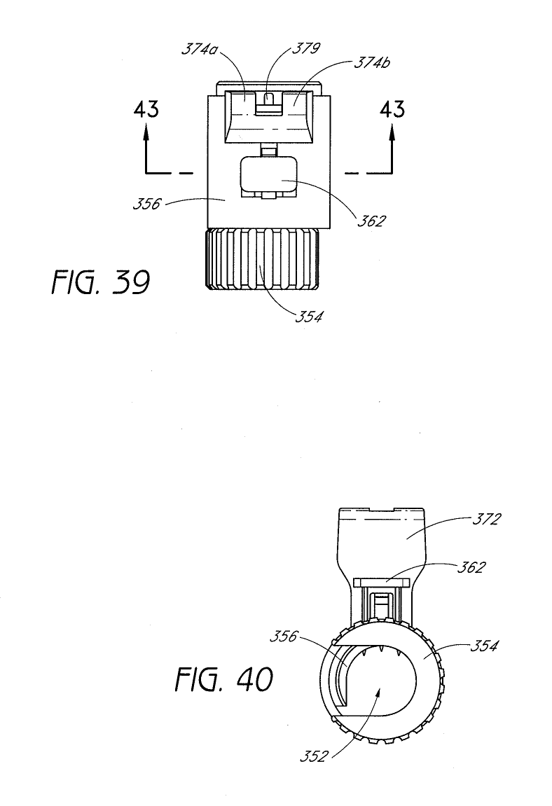

FIGS. 35-43 further illustrate the retainer 350 from FIG. 31. The retainer 350 is configured to receive a portion of an endotracheal tube such that the tube is aligned with a patient's mouth and attach to the support member 320. As can be seen in a front view of the retainer 350 in FIG. 40, the retainer 350 forms a channel 352 that receives at least a portion of an endotracheal tube. The retainer 350 substantially inhibits the endotracheal tube from moving in at least a longitudinal direction when the endotracheal tube is placed in the retainer 350 and the retainer 350 is closed around the endotracheal tube. The retainer 350 preferably further inhibits movement of the endotracheal tube in lateral and transverse directions.

The retainer 350 releasably secures the endotracheal tube. A rotatable collar 354 and a stationary member 356 are configured to form the channel 82 when in a closed position. The rotatable collar 354 is rotatable about the stationary member 356.

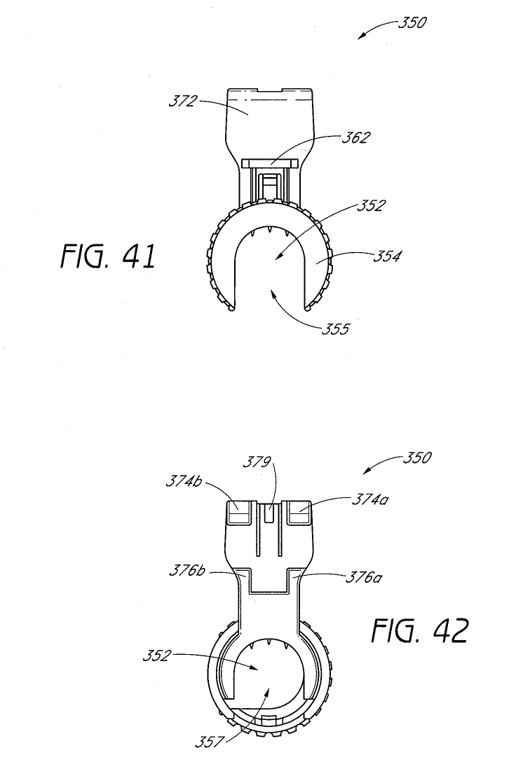

The rotatable collar 354 is configured to accept at least a portion of the endotracheal tube. The rotatable collar 354, however, is not contiguous around an enclosed space. Rather, an opening 355 in the rotatable collar 354 allows ingress of the endotracheal tube into the rotatable collar. The opening 355 is illustrated in a front view of the retainer in FIG. 41.

The stationary member 356 is similarly configured to accept at least a portion of the endotracheal tube. The stationary member 356 is also configured with an opening 357 to allow ingress of the endotracheal tube. The opening 357 is illustrated in a back view of the retainer 350 in FIG. 39.

When the opening 355 and the opening 357 are at least partially aligned, the retainer 350 is in an open position as illustrated in FIG. 41. In this position, the endotracheal tube may be placed at least partially within the channel 352. After the endotracheal tube is positioned at least partially within the channel 352, the rotatable collar 354 may be rotated such that the opening 355 and the opening 357 do not align, thereby placing the retainer 350 into a closed position as illustrated in FIG. 40. When the retainer 350 is in this position, a portion of the endotracheal tube will be substantially enclosed within the channel 352 and movement of the endotracheal tube will be inhibited.

In the illustrated embodiment, the rotatable collar 354 is roughly “C” shaped, comprising approximately 300 degrees of a circle. The shape of the rotatable collar 354 and the size and shape of the opening 355 may, however, be of any size that allows ingress of at least a portion of the endotracheal tube. The size of the opening 355 is smaller than enclosed portions of the stationary member 356. An outer surface of the rotatable collar 354 need not be substantially circular. An outside surface of the rotatable collar 354 may be textured, such as with the splines seen in a side view of the retainer 350 in FIG. 38, to increase the ease with which the medical practitioner may press the grip and manipulate the rotatable collar 354.

In the illustrated embodiment, the stationary member 356 is also roughly “C” shaped and may be configured as a size and shape similar to the rotatable collar 354. The opening 357 may also be configured similar to the opening 355. The opening 357 is not limited to being disposed underneath the retainer 350 as illustrated, and may instead be located on either side of the retainer 350.

The size and shape of the channel 352 defined by the rotatable collar 354 and the stationary member 356 can vary. The channel 352 is illustrated as having a substantially circular shape. The channel 352 may also have another shape, such as rectangular. In one embodiment, the retainer 350 is configured to retain endotracheal tubes from 5 up to a size 10.

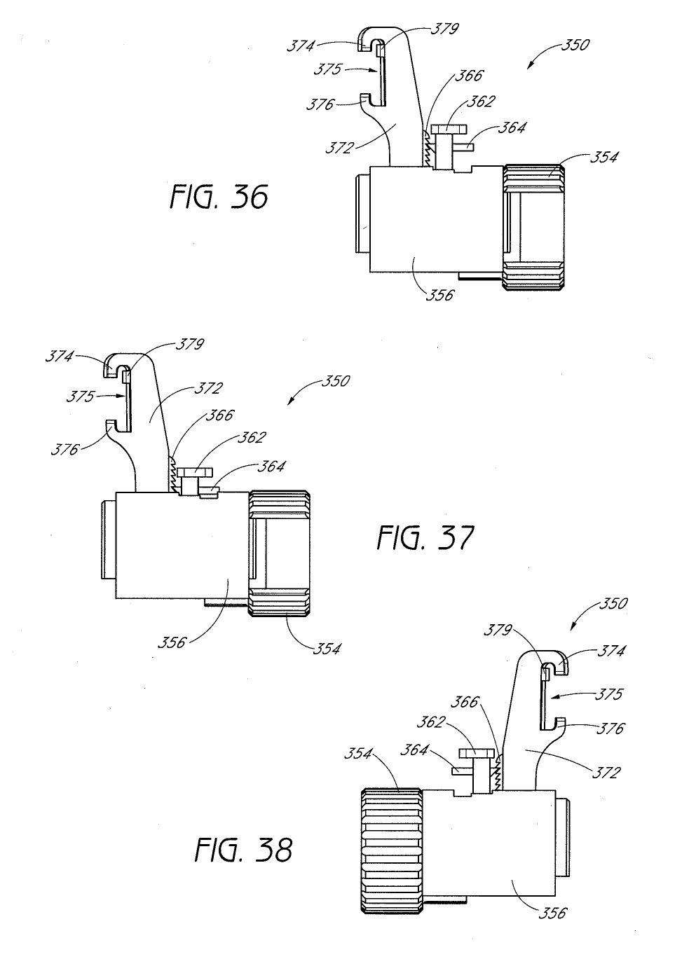

As can be seen in a side view of the retainer 350 in FIG. 36, the retainer 350 includes a depressible member 362. The depressible member 362 has a pawl 364 that engages a ratchet 366. When downward pressure is applied to the depressible member 362, the pawl 364 engages with sequential teeth on the ratchet 366. When pressure is released, the pawl 364 will maintain the depressible member 362 in a depressed position, as can be seen in FIG. 37. To release the depressible member 362, a medical practitioner can apply upward pressure to a distal area of the pawl 364. The depressible member 362 can be maintained in as many positions as there are teeth on the ratchet 366.

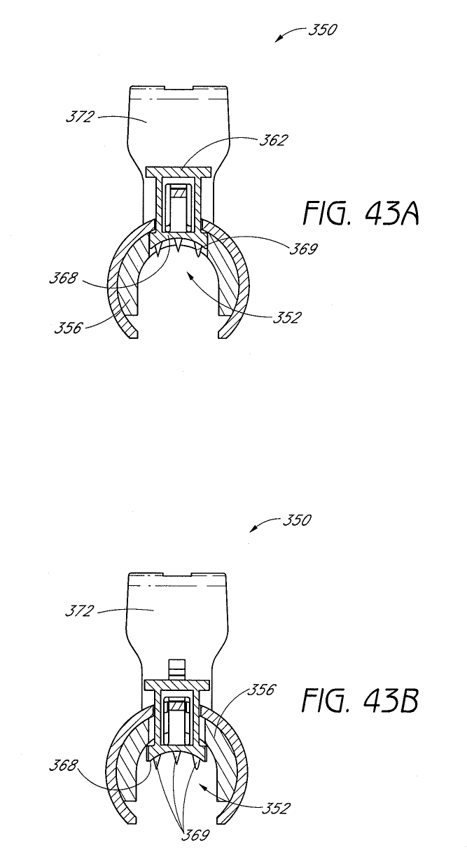

As can be seen in a cross-sectional view of the retainer 350 in FIG. 43A, a lower portion 368 of the depressible member 362 is located within or above the channel 352. When the depressible member 362 is pressed down, the lower portion 368 enters the channel, thereby reducing the area within the channel 352 that an endotracheal tube can occupy, as illustrated in FIG. 43B. When the retainer 350 is in the closed position, the lower portion 368 will pressure the endotracheal tube against the rotatable collar 354, thereby inhibiting movement of the endotracheal tube. Those of skill in the art will appreciate that the retainer 350 can thus be used to secure endotracheal tubes of varying sizes.

The depressible member 362 may include one or more protrusions, friction ridges, or barbs 369 to inhibit longitudinal movement of the endotracheal tube. When the depressible member 362 is pressed down, the barbs 369 may press against the endotracheal tube and hold the endotracheal tube without imparting torque to the endotracheal tube in case the endotracheal tube is pulled in a longitudinal direction. In certain embodiments, each barb has a generally conical shape with a blunt tip. In some embodiments, the barb may extend into the channel 352 by an amount ranging between about 0.1 mm and about 3 mm. Although the depressible member 362 is illustrated as having the barbs 369, the barbs 369 may be omitted.

The depressible member 362 and the rotatable collar 354 may be configured to engage when the depressible member 362 is pushed down. For example, the rotatable collar 354 may extend into the stationary member 356 far enough such that a portion of the rotatable collar 354 is located underneath the pawl 364 or another portion of the depressible member 362. When pushed down, the pawl 364 or other portion may enter one of the depressions between the splines on the rotatable collar 354 or may engage with some other region of the rotatable collar 354 in order to hold the rotatable collar 354 in position. Thus, the rotatable collar 354 would be inhibited from unintentionally rotating.

The retainer 350 includes an arm 372 similar to the arm 102 of the retainer 80, as is most clearly illustrated in FIG. 35. The arm 372 is configured to attach to the track 326. The retainer 350 includes upper and lower protrusions 374 and 376 that define a receiving space 375 configured to accept at least a portion of the track 326.

The retainer 350 differs from the retainer 80 in that no actuator similar to the actuator 108 is illustrated. A center protrusion 379 is still included, as can be seen in a top view of the retainer 350 in FIG. 42. The center protrusion 379 may be inserted into the interstices 328 similar to the way in which the center protrusion 109 can be inserted in the interstices 32 of the support member 20. Placing the center protrusion 379 in one of the interstices 328 in this way will secure the retainer 350 in position on the support member 350.

To reposition the retainer 350, a medical practitioner can raise the retainer 350 relative to the track 356, thereby pulling the center protrusion 379 out of the interstices 328. The retainer 350 may then be translated along the track 326. Of course, the center protrusion 379 may be omitted from the retainer 350.

Those of skill in the art will appreciate that the retainer 350 can thus be moved and repositioned relative to the support member 320, similar to how the retainer 80 can be repositioned relative to the support member 80. Thus, the retainer 350 can be translated along the track 320 without removing the retainer 350, or an endotracheal tube secured by the retainer 350, from the patient.

Although the upper and lower protrusions 374 and 376 are illustrated as comprising left protrusions 374a and 376a, and right protrusions 374b and 376b, the upper and lower protrusions 374 and 376 may each be a single protrusion that may extend across the width of the retainer 350.

The retainer 350 also differs from the retainer 80 in that the retainer 350 does not include a bite block. The retainer 350 may, however, include a bite block similar to the bite block 92. Similarly, the bite block 92 may be omitted from the retainer 80. Other components of the retainer 350 may be configured similar to components of the retainer 80.

Although the retainer 350 does not include a bite block, the securement system 310 does include the bite block 322, as described earlier. In contrast to the bite block 92 of the securement system 10, the bite block 322 is stationary and will not move when the retainer 350 is relocated.

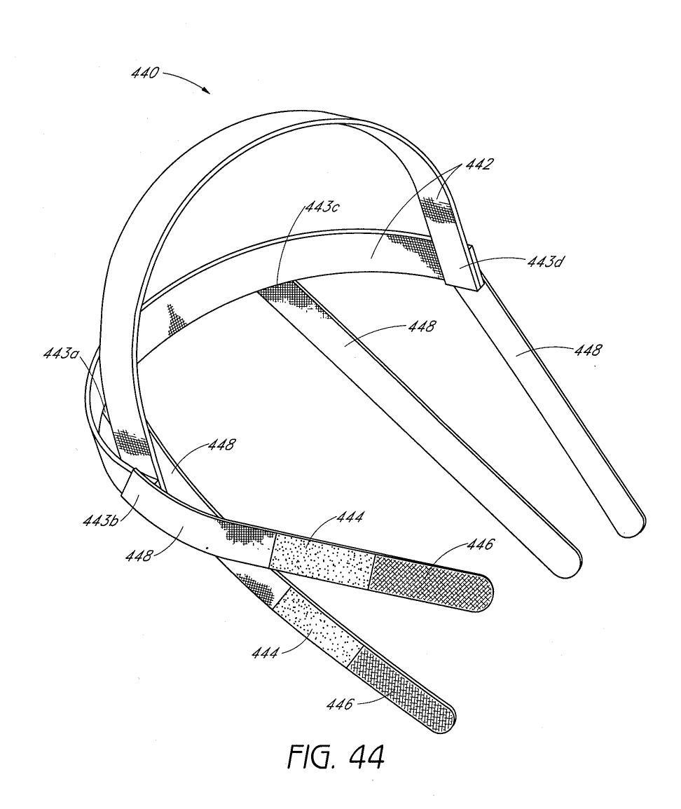

FIG. 44 further illustrates the harness 440 from FIG. 31. The harness 440 may be configured as described in U.S. patent application Ser. No. 11/523,759, filed Sep. 19, 2006, and entitled “ENDO-TRACHEAL TUBE SECUREMENT SYSTEM,” which is hereby incorporated by reference in its entirety.

The harness 440 retains the support member 320 by connection to the connection members 190a-190d, such that the support member 320 is maintained in an operative position to support an endotracheal tube. The harness 440 which secures the support member 320 to the patient's face includes a head contact member 442 and attachment members 448. In the embodiment illustrated in FIG. 44, the head contact member 442 includes securing regions 443a, 443b, 443c, 443d extending from the head contact member 442. The securing regions 443a, 443b, 443c, 443d are spaced around the circumference of the head contact member 442. The embodiment of the head contact member 442 illustrated in FIG. 44 is made of interconnected straps.

Each attachment member 448 comprises a first end portion and a second end portion. The first end portion of each attachment member 448 comprises hook fasteners 444 and loop fasteners 446, which may be configured similar to the hook and loop fasteners 254 and 256 described above in reference to the strap 240. The first end portion of the attachment member 448 may be configured for attachment to at least one of the connection members 190a-190d. Alternatively, the first end portion of one or more attachment members 448 may be configured for attachment directly to the support member 320 or to a connection member different from the connection members 190a-190d illustrated in FIG. 31.

The second end portion of the attachment member 448 is configured for attachment to the securing regions 443a-443d of the strap head contact member 442. Preferably, the second end portion of each attachment member 448 is integral with the strap head contact member 442.

The attachment members 448 may comprise an elastic or otherwise flexible material which keeps the attachment members 448 taut between the back of the patient's head and the support member 320 by stretching or flexing the attachment members 448. For example, the attachment member 448 may comprise VELSTRETCH made by Velcro USA Inc. located in New Hampshire. Alternatively or in combination with an elastic portion, the attachment members 448 may include adjustment means for changing the length of the attachment members 448 between the integral or fixed first or second ends. The adjustment means may be a mechanical system which allows the medical practitioner to change the length of the attachment members 448 between the head contact member 442 and the support member 320. Other adjustment means include but are not limited to: snaps, clips, hook and loop fasteners, or such other fasteners as are known to those of skill in the art.

The strap head contact member 442 generally encircles at least a portion of the patient's skull. In certain embodiments, the strap head contact member 442 has the shape of a closed loop and is sized to receive a portion of the skull of a patient. In certain embodiments, the strap head contact member 442 encircles the lambda of the patient's skull. In certain embodiments, the strap head contact member 442 spans across at least the sagittal suture of the patient's skull. In preferred embodiments, when positioned with the attachment members 448 in tension, the head contact member 442 exerts pressure on the occipital bone, and preferably also on the parietal bone.

Preferably, the loop of the strap head contact member 442 is smaller than the patient's skull and restricts the skull from passing entirely through the center of the loop. While the illustrated embodiment of the strap head contact member 442 has a fixed sized opening, in certain embodiments the strap head contact member 442 includes overlapping ends having suitably, infinitely adjustable fastening means, such as hook and loop fasteners (e.g., VELCRO, fastener pads) whereby the strap head contact member 442 may be adjusted to firmly and snuggly encompass a portion of the patient's head.

The connection members 190a-190d illustrated in FIG. 31 are configured to attach the harness 440 to the support member 320. In the illustrated embodiment, each of the connection members 190b-190d may be configured similar to the connection member 190a, which was described above in reference to FIG. 19. Of course, additional connection members may be added to the securement system 310, for example to connect additional head securement members to the support member 320.

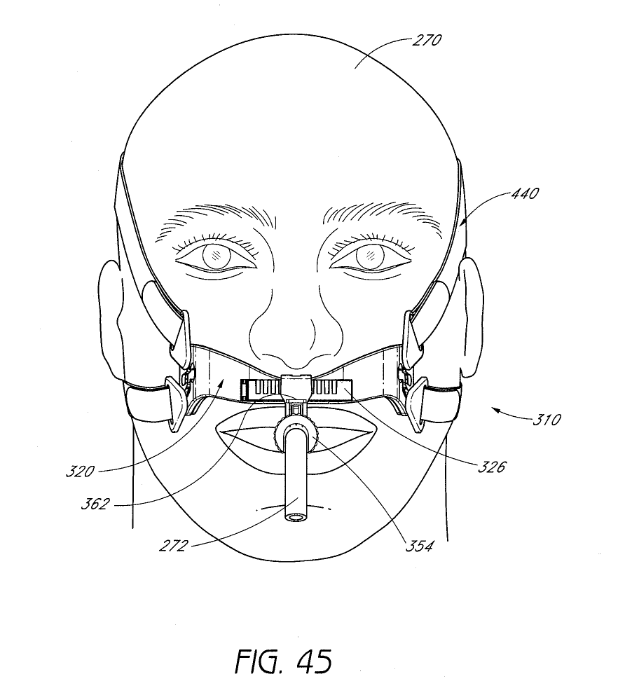

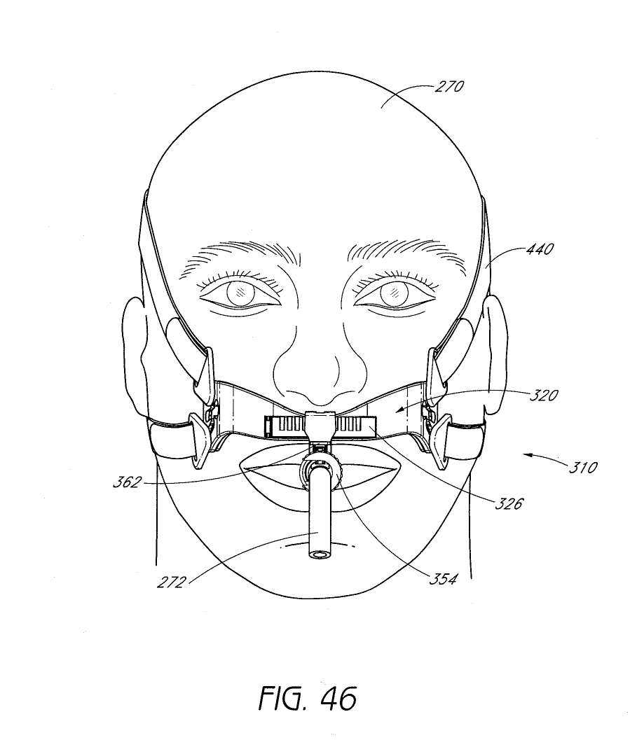

A front view of the intubated patient 270 with the securement system 310 attached is illustrated in FIG. 45. The support member 320 can be attached to the patient 270 using the harness 440 similar to the ways in which the support member 20 can be attached to the patient 270 using the strap 240. The harness 440, however, includes two sets of complimentary hook and loop fasteners 444 and 446 on each side, in contrast to the one set included on each side of the strap 240. The medical practitioner may first attach both sets of hook and loop fasteners 444 and 446 to connection members and/or both connection members to the support member 320 on a single side of the harness 440. Then, the medical practitioner may attach both sets of hook and loop fasteners 444 and 446 and/or both sets of connection members on the other side of the harness 440. Alternatively, the medical practitioner may connect one set of hook and loop fasteners 444 and 446 and/or one connection member on each side of the harness 440 at a time.

After attachment of the support member 320 to the patient 270, the fit and/or position of the securement system 310 may be adjusted using the hook and loop fasteners 444 and 446. In some embodiments, the fit of the strap head contact member 442 may also be adjusted on the patient. The fit and position may also be adjusted by moving the connection members 190a-190d into a different orientation or to a different position. Similar to the securement system 10, the securement system 310 can be adjusted into any number of configurations without being detached from the patient 270.

At any time before, after, or concurrently with attaching the support member 320 to the patient 270 or adjusting the securement system 310 on the patient 270, a medical practitioner can secure the endotracheal tube 272 with the retainer 350. To do so, the medical practitioner places the retainer 350 into an open position, as described above, if the retainer 350 is not already opened. The medical practitioner may then place the retainer 350 over a portion of the endotracheal tube 272 such that the portion is at least partially within the channel 352, as shown in FIG. 45. Subsequently, the medical practitioner can rotate the rotatable collar 354 around the endotracheal tube 272 and secure the retainer 350 in a closed position, as described above. The medical practitioner may then push the depressible member 362 down to secure the endotracheal tube 272, as shown in FIG. 48.

As described above, the position of the retainer 350 on the track 326 may be selected by the medical practitioner. Similar to operation of the securement system 10, the positioning of the endotracheal tube 272 may be secured in a multitude of positions without removing the securement system 310 from the patient 270.

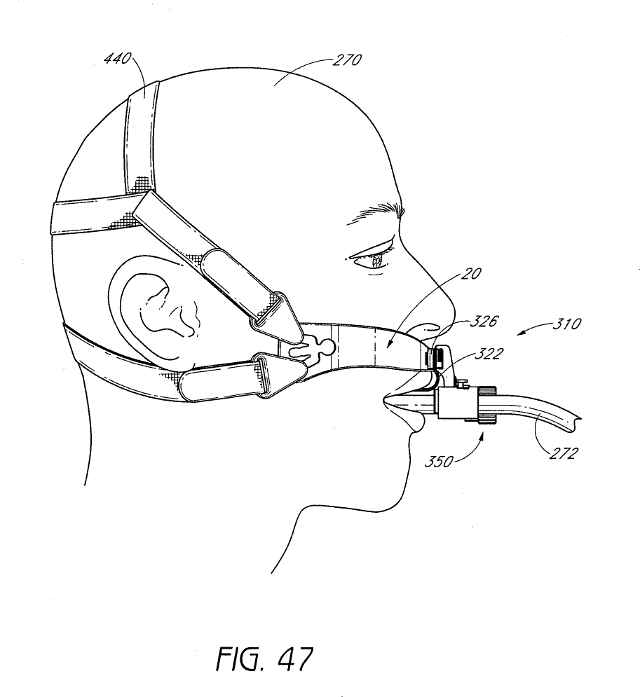

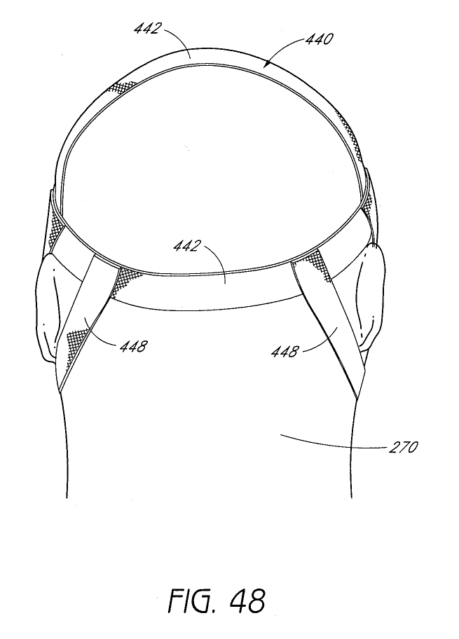

FIG. 47 illustrates a side view of the securement system 310 attached to the patient 270. It can be seen that the harness 440 wraps around the back or the head of the patient 270, thereby securing the endotracheal tube 272 in position. It can also be seen that the bite block 322 will be positioned in the mouth of the patient 270, thereby protecting the endotracheal tube 270. The positioning of the harness 440, including the strap head contact member 442, on the head of the patient 270 can be seen in a back view in FIG. 48.

Similar to the securement system 10, all of the components of the securement system 310 may be packaged and/or assembled in a kit when provided to the medical practitioner. The kit may contain additional components. Of course, the medical practitioner may also assemble various components of the securement system 310.

Those of skill in the art will appreciate that any of the components illustrated in the securement system 10 can be utilized in the securement system 310. For example, the retainer 80 may be used in the securement system 310 in place of the retainer 350. Similarly, any of the components illustrated in the securement system 310 may be utilized in the securement system 10. For example, the harness 440 may be used in place of or in addition to the strap 240.

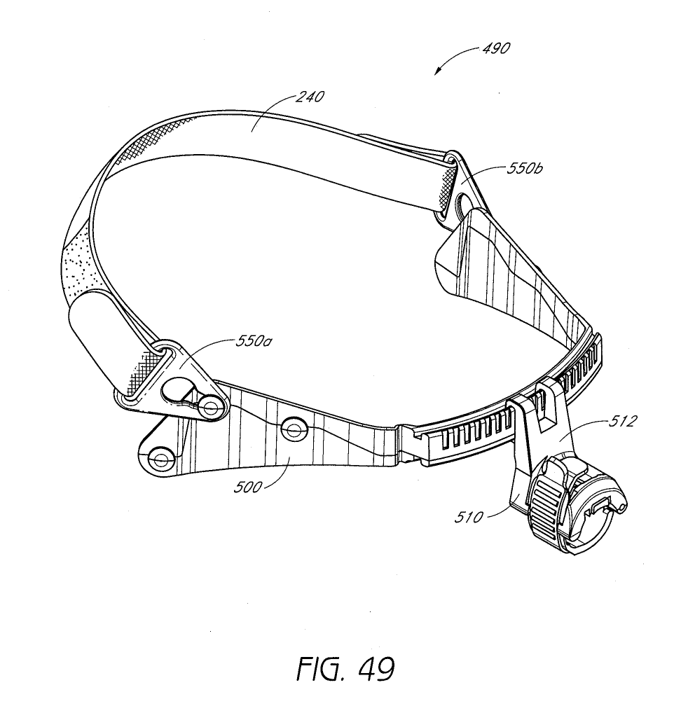

With reference now to FIG. 49, another embodiment of a securement system 490 includes a support member 500, a retainer 510, connection members 550a and 550b, and the strap 240. Similar to the securement systems 10 and 310 illustrated in FIGS. 1 and 31, respectively, the securement system 490 is configured to attach to a patient and secure an endotracheal tube to the patient. The strap 240 is the same as the strap 240 illustrated in FIG. 24.

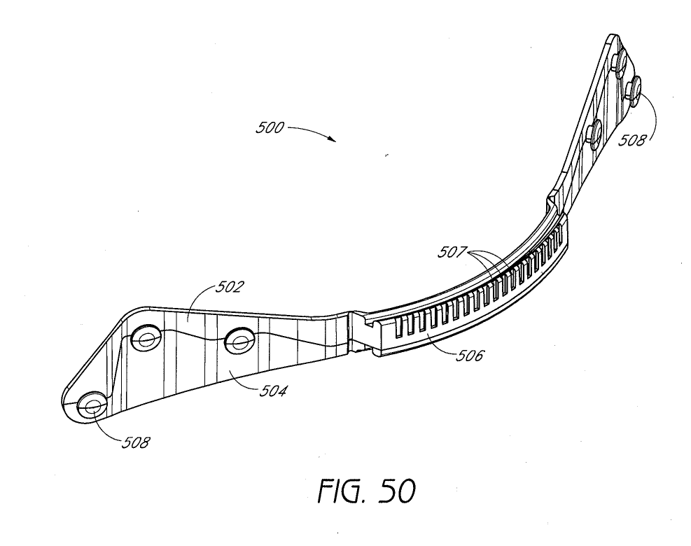

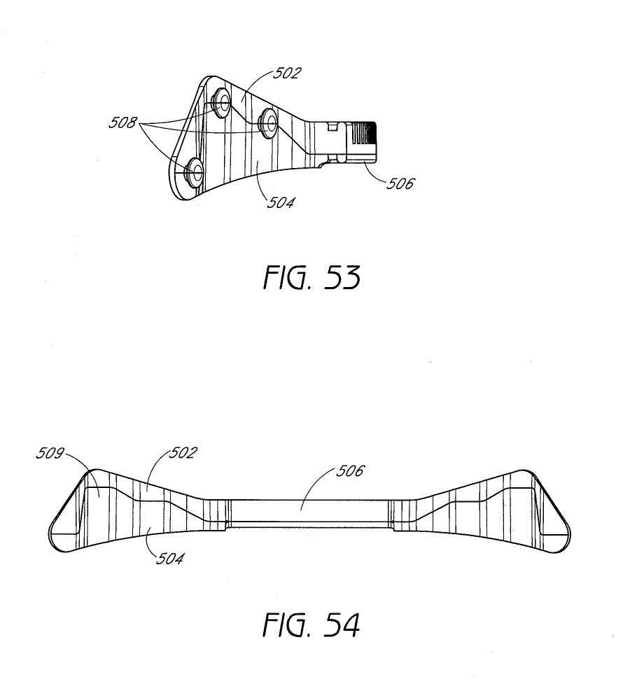

FIGS. 50-54 further illustrate the support member 500. As can be seen in a back view of the support member 500 in FIG. 54, the support member 500 may be formed from a plurality of pieces. In the illustrated embodiment, the support member 500 is formed of a top piece 502 and a bottom piece 504. The top piece 502 and bottom piece 504 may each be manufactured similar to the anterior structure 24 of the securement system 10. The top piece 502 and the bottom piece 504 may be attached in any way known in the art, such as by using a suitable adhesive.

As can also be seen in the back view, a posterior facing surface 509 of the illustrated embodiment of the support member 500 is not overmolded with a soft gel. In other embodiments, the posterior facing surface comprises a soft gel material. Similarly, the support member 20 and the support member 320 may omit the soft gel.

As can be seen in a perspective view of the support member 500 in FIG. 50, a track 506 of the support member 500 is integral to the top piece 502 and bottom piece 504. Thus, the track 506 is not separately formed and then later attached to the support member 500. This integral formation may reduce the amount of material used to form the front of the support member 500. Such reduced amount of material in the front of the support member 500 may improve flexibility of the support member 500. In this way, the support member 500 could be flexed or conformed to fit a wider range of faces of various sizes and shapes.

As can be seen in a front view of the support member 500, interstices 507 are formed in the track 506. The interstices 507 may be configured similar to the interstices 32 of the track 30. Other features of the track 506, such as a groove in the track 506, may also be configured similar to features of the track 30.

The support member 500 includes protrusions 508. The protrusions 508 are configured for insertion into the connection members 550a and 550b. The protrusions 508 allow the connection members 550a and 550b and the strap 240 to be attached to the support member 500.

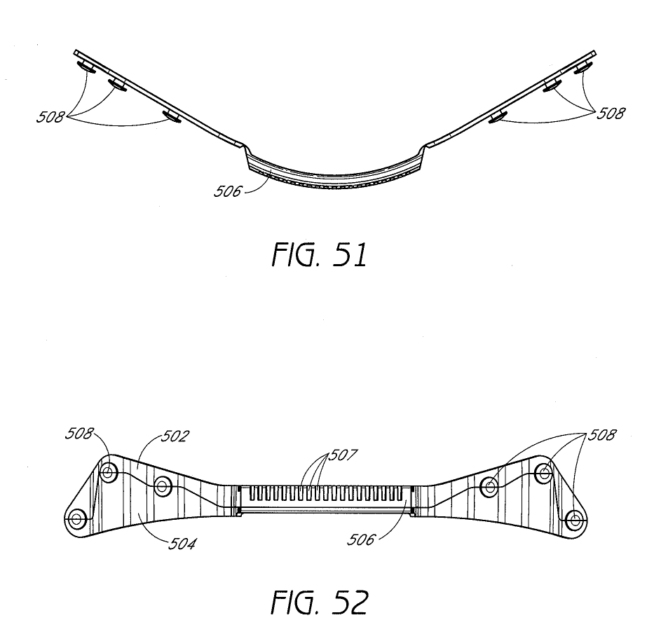

As can be seen in a top view of the support member 500 in FIG. 51, the protrusions 508 may comprise a neck and a head similar to the neck 196 and the head 194 of the connection member 190a. The neck and the head allow for secure attachment of the connection members 550a and 550b to the support member 500, while also allowing the connection members 550a and 550b to rotate.

As can be seen in a side view of the support member 500 in FIG. 53, the protrusions 508 are located at a plurality of transverse and longitudinal locations on the support member 500, which may be referred to as attachment points or locations. This plurality of locations allows a medical practitioner to choose where attachment of the strap 240 would be most beneficial, or allows the medical practitioner to attach multiple straps to the support member 500. Thus, the connection members 550a and 550b can not only rotate or pivot while attached to the support member 500, the point about which they rotate can be chosen.

In the illustrated embodiment, the protrusions 508 are substantially circular. The protrusions 508 may, however, be configured as a different shape. Any of the shapes discussed in reference to the head 194 and or neck 196 of the connection member 190a may be selected for the shape of the protrusion 508.

The support member 500 may otherwise be configured similar to either or both of the support members 10 and 310. Similarly, features of the support member 500 may be utilized in either or both of the support members 10 and 310.

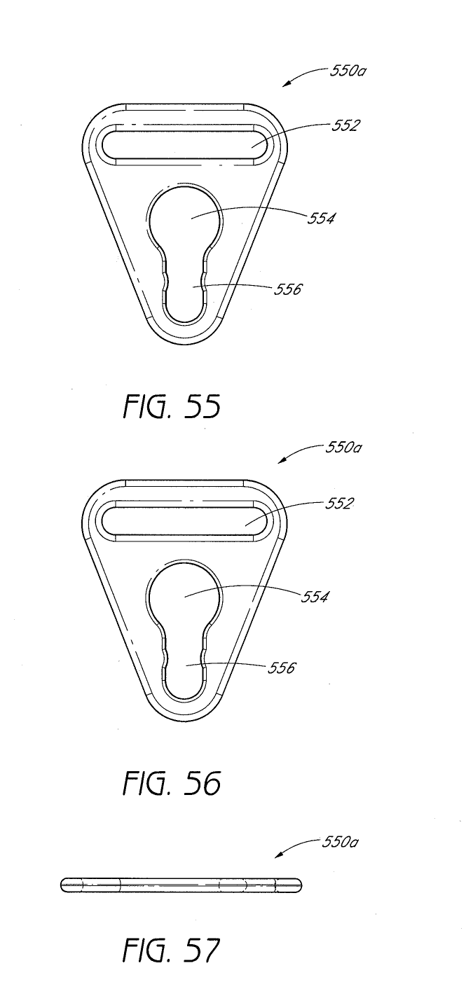

FIGS. 55-57 further illustrate the connection member 550a from FIG. 49. The connection member 550a is configured to attach the strap 240 to the support member 500. The connection member 550a defines a first opening 552 therethrough, as can be seen in a top view and a bottom view of the connection member 550a in FIG. 55 and FIG. 56, respectively. The first opening 552 accepts a portion of the strap 240 and may be configured similar to the opening 192 in the connection member 190a.

The connection member 550a also includes a second opening having a circular opening portion 554 connected to a finger-shaped opening portion 556. The circular opening portion 554 is configured to allow any of the protrusions 508 to be placed through it. The connection member 550a can then be manipulated so that the protrusion 508 slides into the finger-shaped opening portion 556, which may press against the head of the protrusion 508 and prevent the connection member 550a from becoming detached. The connection member 550a will still be able to rotate, however. The circular opening portion 554 and the finger-shaped opening portion 556 may be configured similar to the circular recess 28a and any of the finger-shaped recesses 28b-28d.

The number of openings in the connection member 550a may vary. In some embodiments, the connection member 550a includes only one opening which can accommodate the strap 240 and any of the protrusions 508 concurrently. In other embodiments, the connection member 550a includes a plurality of openings through which any of the protrusions 508 may be inserted.

The connection member 550b may be configured similar to the connection member 550a. The securement system 490 may also include additional connection members, which may be configured similar to the connection member 550b.

The retainer 510 illustrated in FIG. 49 and the retainer 80 illustrated in FIG. 8 do not share the same features. In FIG. 49, it can be seen that the bite block 92 has been omitted, as has the actuator 108 and the center protrusion 109. Thus, the retainer 510 will not lock on the track 506 using the same structure as employed by the retainer 80 to lock on the track 30. If locking is desired, protrusions may be provided on a proximal area of an arm 512 of the retainer 510. The configuration and operation of the retainer 510 may otherwise be similar to that of the retainer 80.

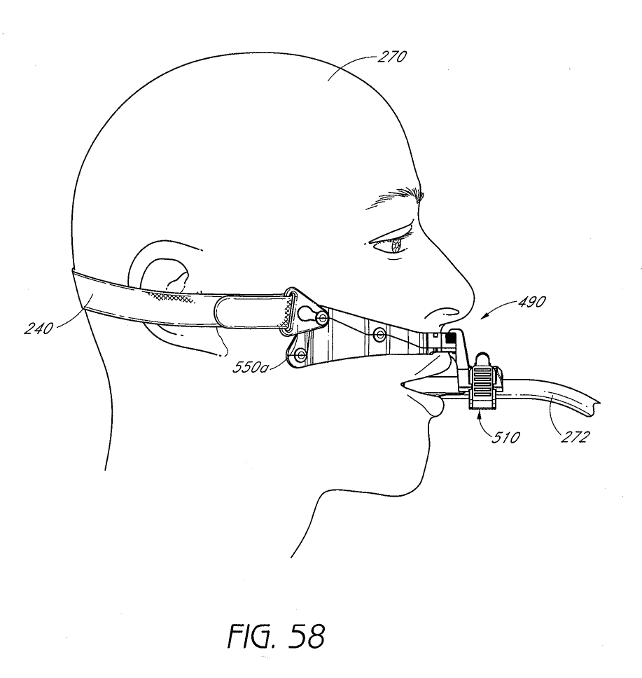

A perspective view of the securement system 490 secured to the patient 270 is illustrated in FIG. 58. A medical practitioner may attach the securement system 490 to the patient 270 similarly to attaching the securement system 10 to the patient 270. Those of skill in the art will recognize, however, that the connection members 550a and 550b, however, are placed over the protrusions 508 instead of portions of the connection members being placed in recesses, as in the securement system 10.

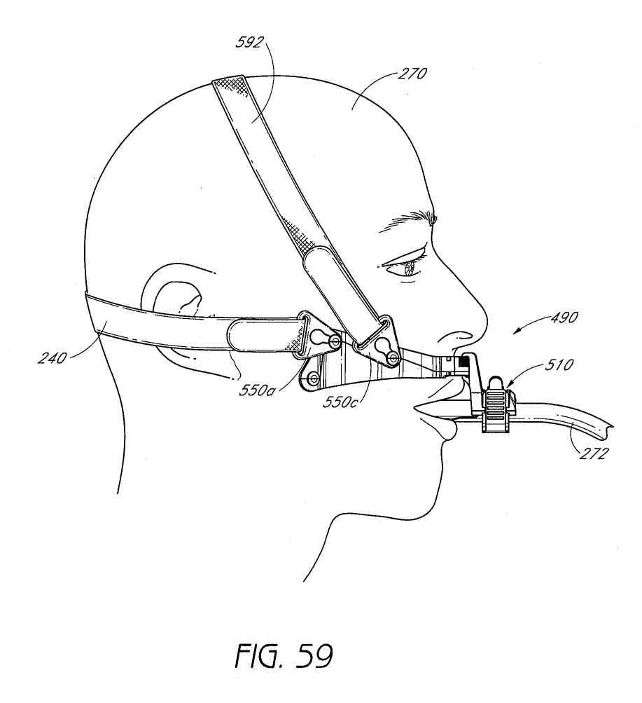

A perspective view of another embodiment of the securement system 490 secured to the patient 270 is illustrated in FIG. 59. The difference in the embodiment illustrated in FIG. 59 is that the securement system 410 is illustrated as including an additional strap 592 connected to additional connection member 550c and 550d. This embodiment is in contrast to the single strap 240 and two connection members 550a and 550b illustrated in FIG. 58. Both of the straps 240 and 592 are configured to be placed around the head and/or neck of the patient 270. The strap 592 may be configured similar to the strap 240. The strap 592 may be placed around a portion of the head and/or neck of the patient 270 different from the position of the strap 240, such as around the crown or the top of the head of the patient 270. The addition of the strap 592 may further decrease the likelihood of substantial movement of the securement system 490 when secured to the patient 270.

Similar to the securement systems 10 and 310, the position of the retainer 510 on the track 506 may be selected by the medical practitioner. The endotracheal tube 272 may thus be translated among a plurality of positions without removing the securement system 490 from the patient 270.

Similar to the securement systems 10 and 310, all of the components of the securement system 490 may be packaged and/or assembled in a kit when provided to the medical practitioner. The kit may contain additional components. Of course, the medical practitioner may also assemble various components of the securement system 490.

Those of skill in the art will appreciate components illustrated in the securement systems 10 and 310 can be utilized in addition to or in place of components in the securement system 490. For example, the retainer 80 may be used in the securement system 490 in place of the retainer 510. Similarly, components illustrated in the securement system 490 may be utilized in the securement systems 10 and 310. For example, the connection members 550a and 550b and the protrusions 508 may be used in place of or in addition to the connection members 190a and 190b and the recess 28.

The various embodiments of the securement systems described above in accordance with the present invention thus provide a means to secure an endotracheal tube or other medical line to a patient and allow access to the oral cavity of the patient when the medical line is secured to the patient. The endotracheal tube can be adjusted or repositioned without removing the entire securement assembly, and without the need for use of additional tape to re-secure the endotracheal tube once it is properly repositioned.

Of course, it is to be understood that not necessarily all objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the disclosure and the claims that follow.

What is claimed is:

1. A securement system for an endotracheal tube comprising:
a support member having a track and a posterior facing surface configured to inhibit movement of the support member into a person's mouth; and
a retainer having a channel configured to receive a portion of the endotracheal tube, the retainer being configured to attach to the track and being movable with respect to the support member in at least one direction.

2. The system of claim 1, wherein at least a portion of the retainer rotates with respect to the support member.

3. The system of claim 1, wherein the retainer is configured to translate in a substantially lateral direction along the track.

4. The system of claim 1 further comprising a locking mechanism configured to inhibit movement of the retainer with respect to the support member.

5. The system of claim 4, wherein the retainer is configured to lock in at least a first position and a second position different than the first position.

6. The system of claim 1 further comprising a bite block configured to move with respect to the support member concurrently with the retainer moving with respect to the support member.

7. The system of claim 1 further comprising at least one protrusion configured to be selectively movable into the channel.

8. The system of claim 7 further comprising a locking mechanism configured to substantially maintain the protrusion at a position in the channel.

9. The system of claim 7, wherein the protrusion is configured to inhibit movement of the endotracheal tube in at least one direction.

10. The system of claim 1 further comprising a plurality of connection members configured to attach to the support member at any of a plurality of attachment points.

11. The system of claim 10, wherein the connection members rotate about the attachment points such that the connection members may be disposed at multiple angles of rotation about the attachment point.

12. The system of claim 1 further comprising a soft gel in contact with at least a portion of the patient's face.

13. A securement system for an endotracheal tube, the system comprising:
a track; and
a retainer configured to receive a first portion of the endotracheal tube so as to inhibit movement of the endotracheal tube in at least one direction while a second portion of the endotracheal tube is placed in a patient's mouth, the retainer being attached to the track so as to translate along the track without being removed from the track.

14. The system of claim 13 further comprising a bite block configured to translate along the track.

15. The system of claim 13, wherein the track is configured to define a plurality of locations at which the retainer can be secured.

16. The system of claim 13, wherein the retainer is configured to receive and secure endotracheal tubes of sizes 5-10.

17. The system of claim 13 further comprising a movable portion configured to secure endotracheal tubes of a plurality of sizes at least partially within the retainer.

18. The system of claim 17, wherein the movable portion comprises a pivoting member configured to define a channel at least when placed in a closed position.

19. The system of claim 17, wherein the movable portion comprises a depressible member configured to secure the endotracheal tube against a portion of the retainer.

20. The system of claim 19 further comprising a pawl and ratchet configured to maintain a position of the depressible member.

21. The system of claim 13 further comprising a channel and a collar rotatable about the channel, wherein the collar is configured to allow ingress of the first portion of the endotracheal tube into the channel when the collar is in at least a first position, and wherein the collar is configured to prevent egress of the first portion of the endotracheal tube from the channel when the collar is in at least a second position.

22. The system of claim 21 further comprising a lock configured to secure the collar in at least the second position.

23. The system of claim 13 further comprising a plurality of recesses and at least one protrusion, the protrusion being configured to secure the retainer in position on the track when inserted into any of the recesses.

24. The system of claim 13 further comprising a soft gel configured to contour to a shape of the patient's face.

25. The system of claim 24, wherein the soft gel comprises a material impregnated with medical grade mineral oil.

26. A method of securing an endotracheal tube in a first position on a patient so as to facilitate access to the patient's oral cavity, comprising:
providing an endotracheal securement device having a support member and a retainer, the retainer defining a channel and being movable with respect to the support member;
placing a portion of the endotracheal tube in the channel; and
rotating at least a portion of the retainer so as to secure the portion of the endotracheal tube in the channel.

27. The method of claim 26 further comprising moving the retainer from a first position to a second position relative to the support member so as to facilitate access to the patient.

28. The method of claim 27 further comprising locking the retainer in the second position.

29. The method of claim 26 further comprising causing a plurality of protrusions to move within the channel and contact the portion of the endotracheal tube.

30. The method of claim 29 further comprising locking the plurality of protrusions when at least one of the protrusions are in contact with the portion of the endotracheal tube.
